(19) United States
(12) Patent Application Publication
(10) Pub. No.: US 2005/0143290 A1
(43) Pub. Date: Jun. 30, 2005

(54) Substituted inositols and their use

(76) Inventors: Thomas William Rademacher, Oxford (GB); Richard Schmidt, Konstanz (DE); Andreas Stadelmaier, Mutlangen (DE)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/504,605

(22) PCT Filed: Feb. 13, 2003
(86) PCT No.: PCT/GB03/00604

(30) Foreign Application Priority Data
Feb. 14, 2002 (GB) 0203535.0

(51) Int. Cl. A61K 38/16; C08B 33/02

(52) U.S. Cl. 514/7; 514/25; 530/332; 536/108

(57) ABSTRACT

Inositol phosphate esters and conjugates formed between the compounds and a coupling partner are disclosed, in particular compounds based on a myo-inositol which is substituted at position 1 with a phosphate ester group, at position 2 with a sugar group and at position 4 and/or position 6 with an amino acid group. The compounds are based on the structure of phosphatidylinositol hexamannosides (PIM6) of Mycobacteria and may be used as mimics of the naturally occurring PIMs in order to induce biological responses normally attributed to the natural compound or may be used as biologically inert carriers in order to deliver specific pharmaceutically active compounds to lipid rafts/caveolae.

### FIELD OF THE INVENTION

The present invention relates to substituted inositols and their uses, and in particular to compounds which are based on the structure of phosphatidylinositol hexamannoside (PIM₆) and the uses of conjugates of these compounds and a coupling partner, e.g. for delivery of the coupling partner to caveolae.

### BACKGROUND OF INVENTION

Caveolae are a specialised form of lipid raft, a dynamic assembly of cholesterol and sphingolipids in the plasma membrane. The proposed function of lipid rafts/caveolae is diverse including cholesterol transport, endocytosis, potocytosis and signal transduction. Important cell signalling proteins such as nitric oxide synthase, Ras related GTP-binding proteins are examples of cell-signalling components found in lipid raft/caveolae. Mycobacterial lipoglycans containing a phosphatidylinostitol moiety linked to an oligomannose core glycan (lipoarabinomannans or LAMs) and GPI precursors of the insulin-mimetic inositolphosphoglycans (IPG) also localise to caveolae.

Glycosylphosphatidylinositol (GPI) anchored proteins, localise to caveolae as well as a number of membrane associated phospholipases such as GPI-PLD. It is also believed that endotoxin, a bacterial lipoglycan inserts directly into lipid raft/caveolae.

A substructure of the LAMs called phosphatidylinositol hexamannoside (PIM₆) the GPI-anchor of all LAMs is able to inhibit LAM insertion into lipid rafts/caveolae. Structures based on PIM₆, therefore, contain the necessary structural characteristics to target to lipid rafts/caveolae. A structure for PIM₆ is proposed in J. Immun. 155: page 1334-1342 (1995).

### SUMMARY OF INVENTION

Following the insertion of LAMs and PIM₆ into host membranes, a profound biological response can be elicited. Such responses include induced expression and secretion of TNF-α and IL-6 and inhibition of T-Cell proliferative responses. LAMs have also been shown to inhibit expression of IL-2, IL5, and GM-CSF genes in human T cells and the IFN-γ-mediated activation of macrophages.

Broadly, the present invention relates to compounds based on the structure of phosphatidylinositol hexamannosides (PIM₆) of Mycobacteria, and in particular to compounds which are inositol phosphate esters and conjugates formed between the compounds and a coupling partner. These compounds may be used as mimics of the naturally occurring PIMs in order to induce biological responses normally attributed to the natural compound or may be used as biologically inert carriers in order to deliver specific pharmaceutically active compounds to lipid rafts/caveolae. Thus, in the preferred embodiments, the compounds have the property of localising in caveolae present on the surface of cells, making them useful for delivering the coupling partners to the caveolae. In a further preferred embodiment, the compounds have the property of mimicking the action of the GPI-anchor of the lipoarabinomannans, following localisation of the compounds to lipid rafts/caveolae. In a further preferred embodiment, the compounds have the property of acting as cross-reacting antigens against the LAM GPI anchor components present in mycobacteria and therefore, could afford protection as a vaccine against these diseases. In a further embodiment, these compounds would be useful for delivering the coupling partners to antigenically responsive cells giving rise to an enhanced immune response to the coupling partner. For example, in optimising carbohydrate based/anti-cancer vaccines where carrier structure adjuvant and epitope clustering influence the antibody responses (see PNAS 98: 3264-3269, (2001)).

Accordingly, in a first aspect, the present invention provides a compound comprising a myo-inositol which is substituted at position 1 with a phosphate ester group, at position 2 with a sugar group and at position 4 and/or position 6 with an amino acid group,
or a coupling partner or a derivative of the compound.

Thus, the present invention surprisingly discloses that the hexamannoside group from position 6 of mycobacterial PIM₆ is not a structural motif that is required to obtain the activity of PIM₆ in localising in caveolae. This opens up the possibility of using the compounds of the invention to make conjugates with coupling partners and using these conjugates to deliver the coupling partners to caveolae, for effect in these organelles or for subsequent internalisation into cells.

In a preferred embodiment, the present invention provides a compound, or a coupling partner or derivative thereof, represented by one of the structural formulae: 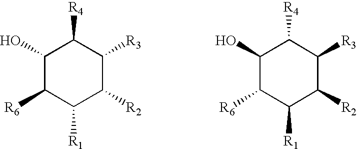
wherein:
R₁ is hydroxyl, phosphate, phosphatidic acid or a phosphate ester;
R₂ is a sugar moiety;
R₃ is are selected from hydroxyl or phosphate; and,
R₄ and/or R₆ is or are independently selected from:
   an amino acid; or
   a peptide or polypeptide; or
   a group having the general formula:
      —O—(CH₂)ₙ—CH (NR₇R₈)—CO₂X,
   wherein:
      n is an integer between 1 and 10,
      R₇ and R₈ are independently selected from hydrogen, nitrogen, acyl or alkyl; and
      X is hydrogen, alkyl or a cation where the terminal group is —CO₂⁻; or
   a substituted or unsubstituted aromatic group, such as a group represented by the general formula: 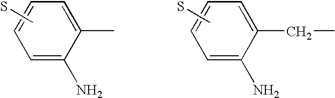
   wherein S is hydrogen or one more aromatic substituents; and
   wherein when one of R₄ or R₆ is as defined above, the other may be hydroxyl or phosphate.

In the above definition, the alkyl groups are preferably C₁₋₁₀ alkyl and may be unsubstituted or substituted and straight chain or branched alkyl. Preferred alkyl groups are methyl, ethyl or propyl groups.

The S groups present in certain R₄ and/or R₆ substituents represents one or more aromatic substituents, for example alkyl, halogen, nitro etc.

The aromatic groups used to mimic sugar residues can be introduced into the compounds of the invention using the corresponding 2-nitro derivatives:

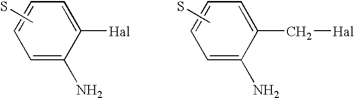

An example of the use of aromatic rings to mimic sugar residues is shown in Muller et al, Angew. Chem. Int. Ed., 1998, 37, 2893-2897.

The myo-inositol can be the D or L enantiomer. L-myo inositols are shown in the formula on the left, while D-myo-inositols are shown on the right. Optionally, the inositol is further substituted with one or more further substituents at position(s) other than the position of linkage to the phosphate ester group, the position of linkage to the sugar group or the position of linkage of the amino acid or the coupling partner.

In some embodiments, the R₁ group is a phosphate ester group which is a phosphate lipid ester in which a phosphate group is linked to position 1 of the inositol ring, the phosphate group being substituted with an alkyl group linked to one or more lipid groups, e.g. having a formula represented by:

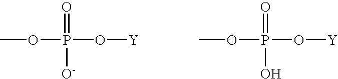

Preferably, the above formula represents a phosphate ester comprises one or more lipidic groups. Examples of lipidic groups include lyso, acyl, alkyl, diacylglyceryl, alkylacylglyceryl, dialkylglyceryl, ceramidyl, lysospingosine, acylglyceryl, or alkylglyceryl groups. Preferred examples of the Y group of the phosphate lipid esters include:
(1) a diacylglyceryl group represented by: 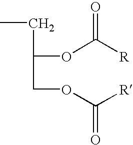
wherein R and R′ are alkyl or substituted alkyl groups, and more preferably are independently selected from C₇ to C₂₈ alkyl or substituted alkyl groups.
(2) an alkyl-acylglyceryl group represented by: 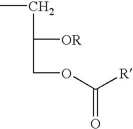
wherein R and R′ are alkyl or substituted alkyl groups, and more preferably are independently selected from C₇ to C₂₈ alkyl or substituted alkyl groups.
(3) a dialkylglyceryl group represented by: 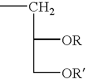
wherein R and R′ are alkyl or substituted alkyl groups, and more preferably are independently selected from C₇ to C₂₈ alkyl or substituted alkyl groups.
(4) a ceramidyl group represented by: 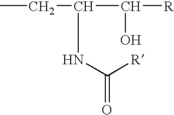
wherein R is a saturated or unsaturated alkyl or substituted alkyl group, for example a C₇ to C₂₈ alkyl or substituted alkyl group, and more preferably is represented by the formula —═CH—R″, —CH₂—CH₂—R″ or —CH(OH)—CH₂—R″, wherein R″ is a C₃ to C₁₇ alkyl or substituted alkyl group and R′ is an alkyl or substituted alkyl group, more preferably independently selected from a C₇ to C₂₈ alkyl or substituted alkyl group.

Specific examples of lipidic moieties include 1-O-(C16:0)lyso-alkylglycerol; (C16:0))lyso-acylglycerol; (C18:0))lyso-acylglycerol; (C20:0))lyso-acylglycerol; (C22:0))lyso-acylglycerol; ceramide, (C16:0)fatty acid-(C18:1)sphingosine; ceramide, (C16:0)fatty acid-(C18:0)sphinganine; ceramide, (C24:0)fatty acid-(C18:1)sphingosine; ceramide, (C24:0)fatty acid-(C18:0)sphinganine; 1-O—(C16:0)alkyl-2-O—(C16:0)acylglycerol; 1-O—(C16:0)alkyl-2-O—(C18:2)acylglycerol; 1-O—(C16:0)alkyl-2-O—(C18:1)acylglycerol; 1-O—(C16:0)alkyl-2-O—(C18:0)acylglycerol; (C16:0)-alkyl-(C16:0)acyl-glycerol (AAG) and (C16:0)mono(lyso)-alkyl-glycerol (MAG).

Preferably, the sugar moiety (R₂) at position 2 is a hexose, and more preferably is selected from glucosamine, galactosamine, galactose, mannose, glucose, fucose or xylose including substituted derivatives thereof. A preferred sugar group is mannose, and more preferably alpha-D-mannose. The sugar group may be optionally substituted at one, two, three, or four positions other than at the position of linkage to the cyclitol moiety (the anomeric position). Examples of sugar group substituents include CF₃, X(CH₂)ₙ—O— (where X is hydrogen, or substituted or unsubstituted alkyl), or CHF₂O—.

The linkage between the inositol and the sugar group is preferably via one of the oxygen atoms of the inositol. However, this oxygen atom can be replaced by another atom or a linker group, e.g. by one or more —CH₂— or —S— groups. The linkage of the sugar residue to the inositol may be in either the α or β configuration. In some embodiments, the R₄ substituent at position 4 and/or the R₆ substituent at position 6 may be an amino acid or amino acid mimetic group or group for linking to a coupling partner, such as a peptide or polypeptide. Examples of amino acids include alanine, isoleucine, leucine, methionine, phenylalanine, proline, tryptophan, valine, asparagine, cysteine, glutamine, glycine, serine, threonine, tyrosine, arginine, histidine, lysine, aspartic acid and glutamic acid, and substituted forms thereof.

Preferred coupling partners are peptides, polypeptides or carbohydrates, and the compounds of the invention can be used to deliver the coupling partner or partners to caveolae. Once at the caveolae the coupling partner(s) may act at the caveolae or may be internalised into cells. Examples of coupling partners include polypeptides, or carbohydrates having activity as vaccines, growth factors, or receptors. The linkage may be via the amino or carboxy terminus of the amino acid or to a side chain. A preferred amino acid linker is serine, e.g. a serine residue coupled via its hydroxyl side chain. In some instances, the coupling partner can also be provided with a serine residue for linkage to the serine residue present on the 4 and/or 6 position of the inositol ring.

By providing a Ser-Ser linkage between a compound of the invention and the coupling partner, one the compound has been delivered to caveolae, enzymatic processing can lead to the cleavage of the Ser-Ser bond, allowing the uptake of the coupling partner.

The coupling partners may be linked to the inositol ring via position 4, position 6 or both positions 4 and 6. In the latter case, it would be possible to employ the compounds of the present invention to deliver two different coupling partners to caveolae, and providing the delivery of a bifunctional conjugate.

Exemplary compounds and intermediates of the invention include those listed below in the examples and especially:
36 D: Triethylammonium-[2-O-(α-D-mannopyranosyl)-D-myo-inosit-1-yl]-[(2R)-2,3-bis-(myristoyloxy)-propyl]-phosphate.
St 30: Triethylammonium-(2-O-(α-D-mannopyranosyl)-L-myo-inosit-1-yl]-[(2R)-2,3-bis(myristoyloxy)propyl]-phosphate.
St 66: 6-O-[(2R)-2-amino-propionic acid]-2-O-α-D-mannopyranosyl-L-myo-inosit-1-yl-[(2R)-2,3-bis-(myristoyloxy)-propyl]-phosphate.
St 50-1: 6-O-[(2S)-2-amino-propionic acid]-2-O-α-D-mannopyranosyl-D-myo-inosit-1-yl-[(2R)-2,3-bis-(myristoyloxy)-propyl]-phosphate.
St 50-2: 6-O-[(2R)-2-amino-propionic acid]-2-O-α-D-mannopyranosyl-D-myo-inosit-1-yl-[(2R)-2,3-bis-(myristoyloxy)-propyl]-phosphate.

The structures of these compounds and the PIM₆ anchor are provided below.

In a further aspect, the present invention provides the novel intermediates disclosed in the syntheses described herein.

In a further aspect, the present invention provides the above compounds for use in a method of medical treatment.

In a further aspect, the present invention provides the use of a compound as defined herein for the preparation of a medicament for the treatment of a condition that responds to the coupling partner or a metabolic product thereof.

By way of example and not limitation, embodiments of the present invention will now be described in more detail with reference to the accompanying figures.

### BRIEF DESCRIPTION OF THE FIGURES

FIG. 1: Synthesis of triethylammonium-[2-O-(α-D-mannopyranosyl)-D-myo-inosit-1-yl]-[(2R)-2,3-bis-(myristoyloxy)-propyl]-phosphate (36 D). Ref.: [11]-[16].

FIG. 2: Synthesis of enantiomerically pure inositol. Ref.: [1]-[6].

FIG. 3: Synthesis of 6-Q-((2S)-2-amino-propionic acid]-2-O-α-D-mannopyranosyl-D-myo-inosit-1-yl-[(2R) -2,3-bis-(myristoyloxy)-propyl]-phosphate (St 50-1) and 6-O-[(2R)-2-amino-propionic acid]-2-O-(-D-mannopyranosyl-D-myo-inosit-1-yl-[(2R)-2,3-bis-(myristoyloxy)-propyl]-phosphate (St 50-2): Ref.: [10]-[15], [17].

FIG. 4: Synthesis of 6-O-[(2R)-2-amino-propionic acid]-2-O-α-D-mannopyranosyl-L-myo-inosit-1-yl-[(2R)-2,3-bis-(myristoyloxy)-propyl]-phosphate (St 66). Ref.: [7]-[15].

FIG. 5: Synthesis of triethylammonium-(2-O-(α-D-mannopyranosyl)-L-myo-inosit-1-yl]-[(2R)-2,3-bis(myristoyloxy)propyl]-phosphate (St 30). Ref.: [13]-[15].

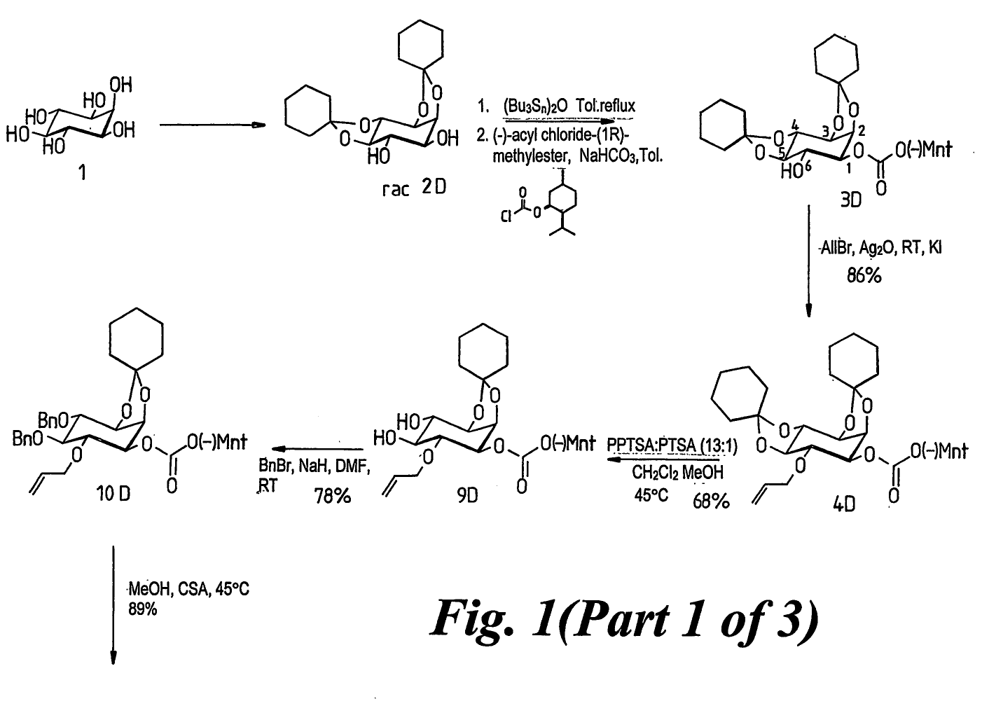

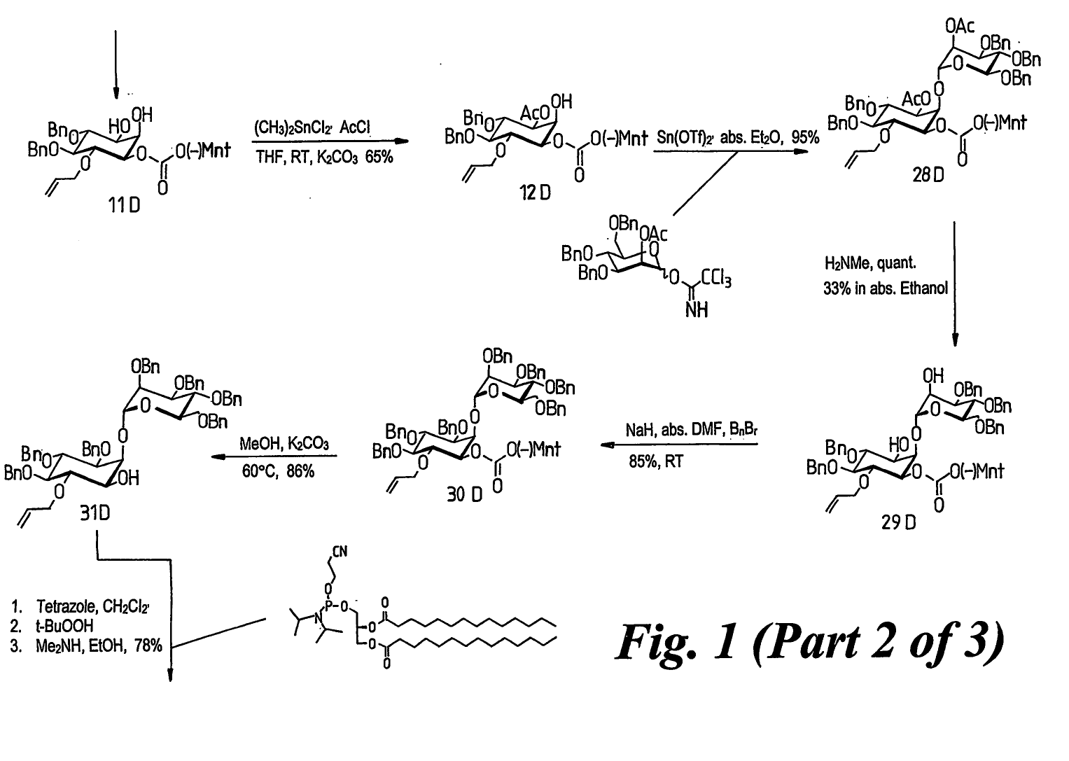

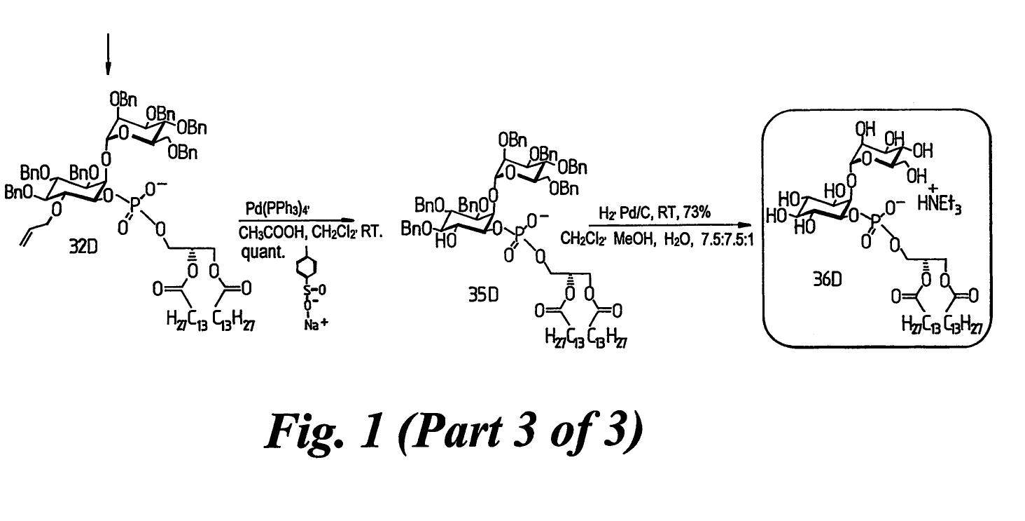

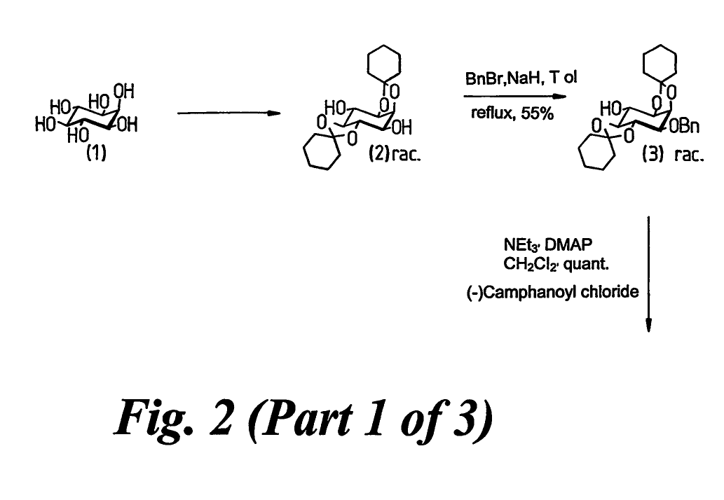

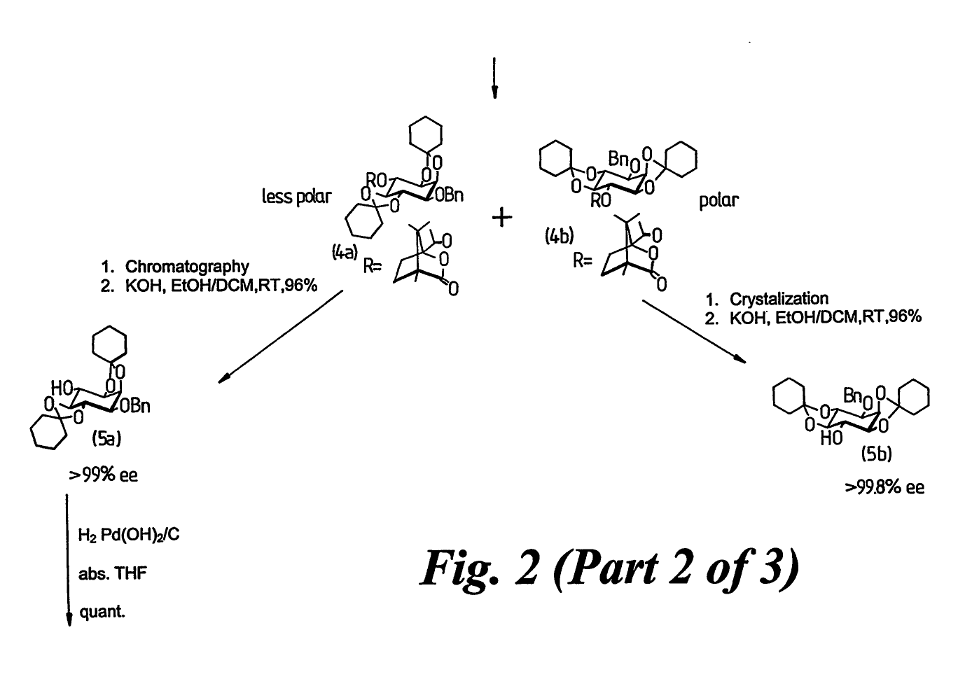

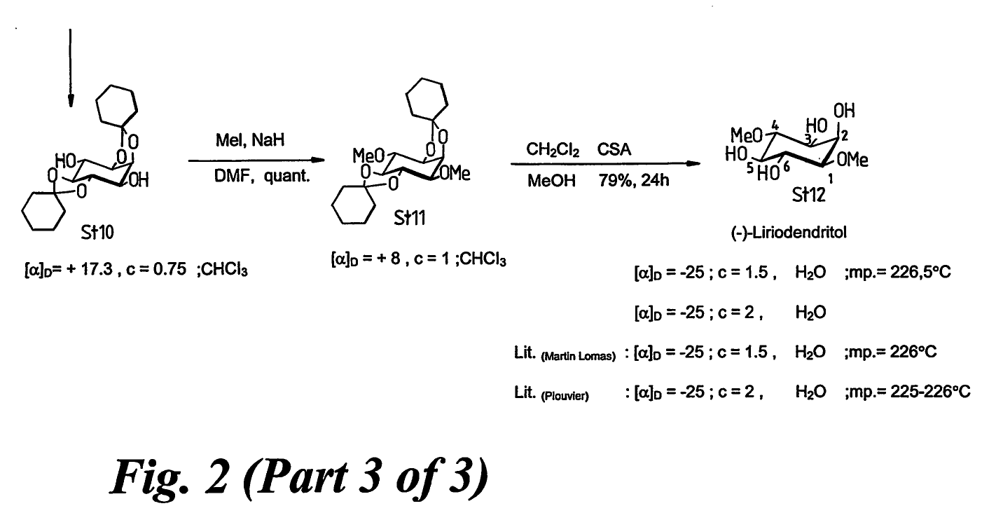

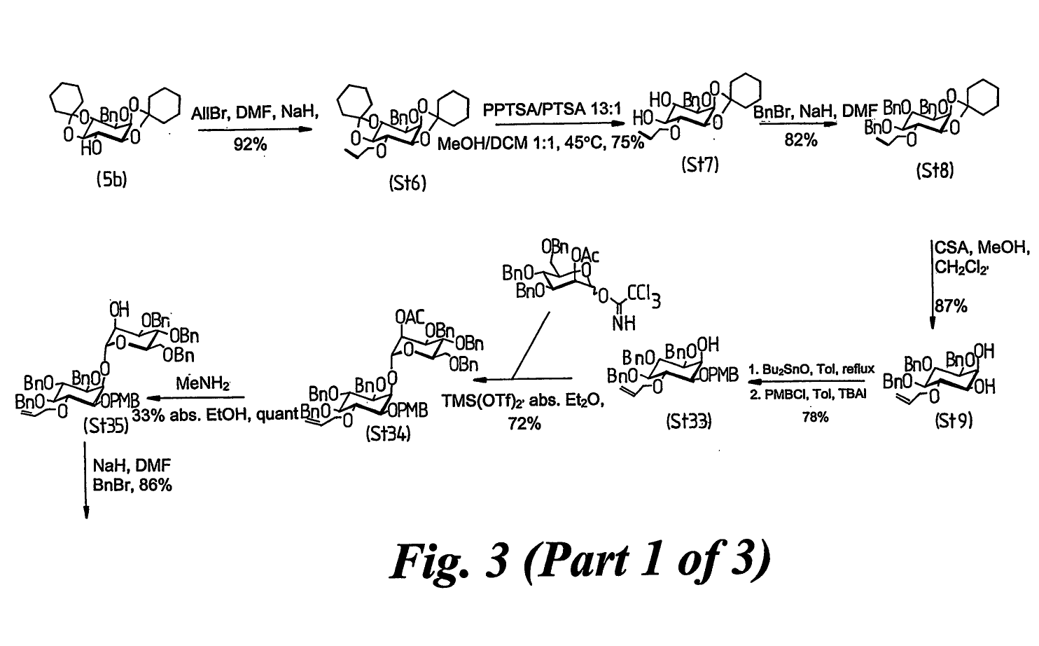

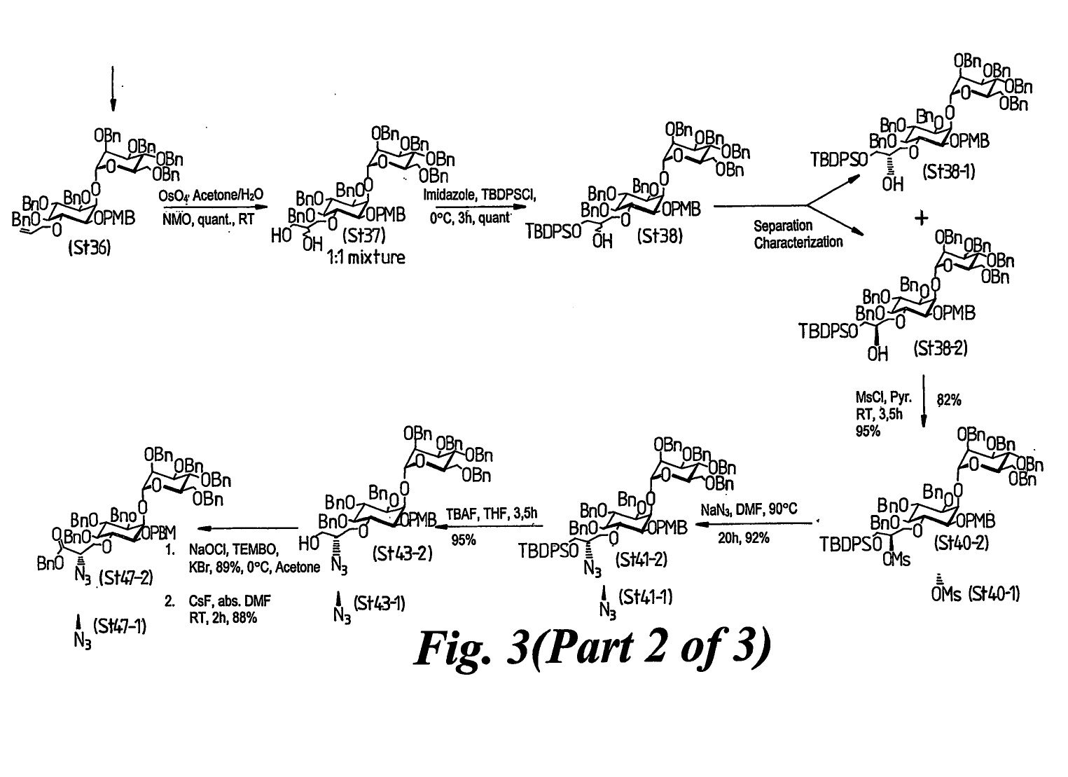

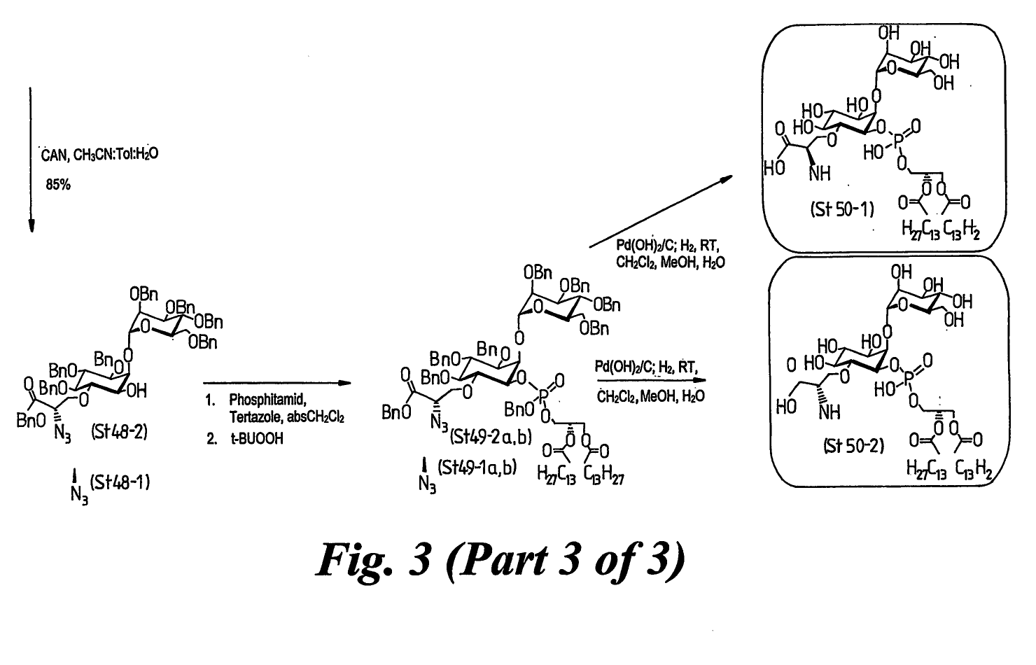

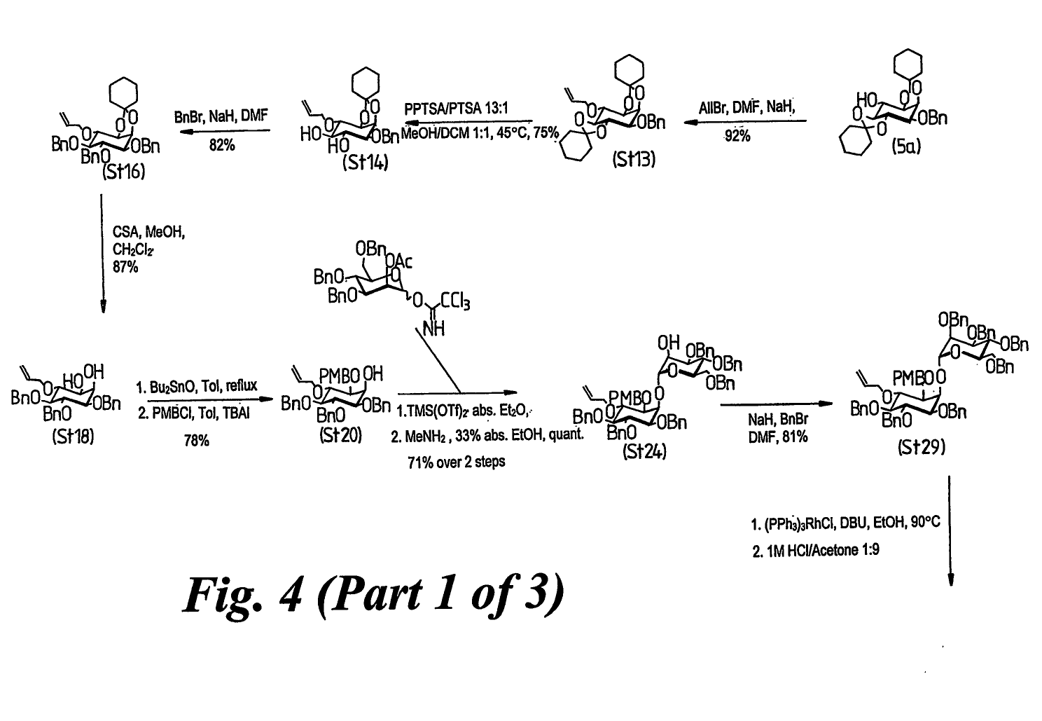

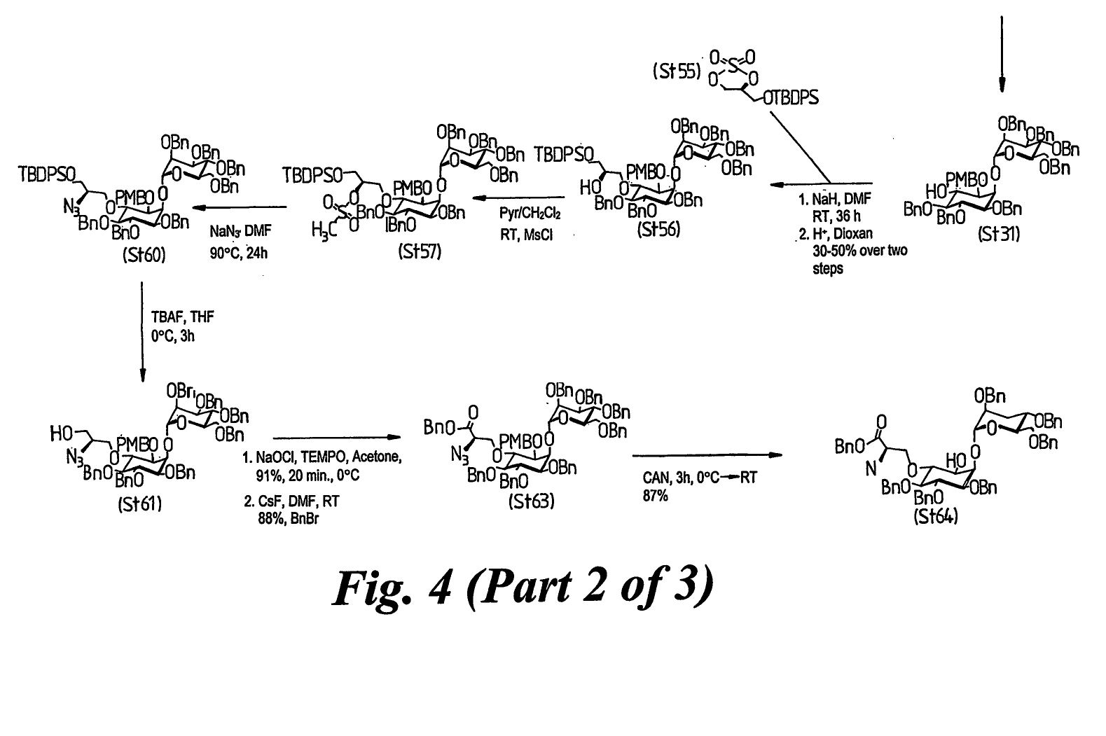

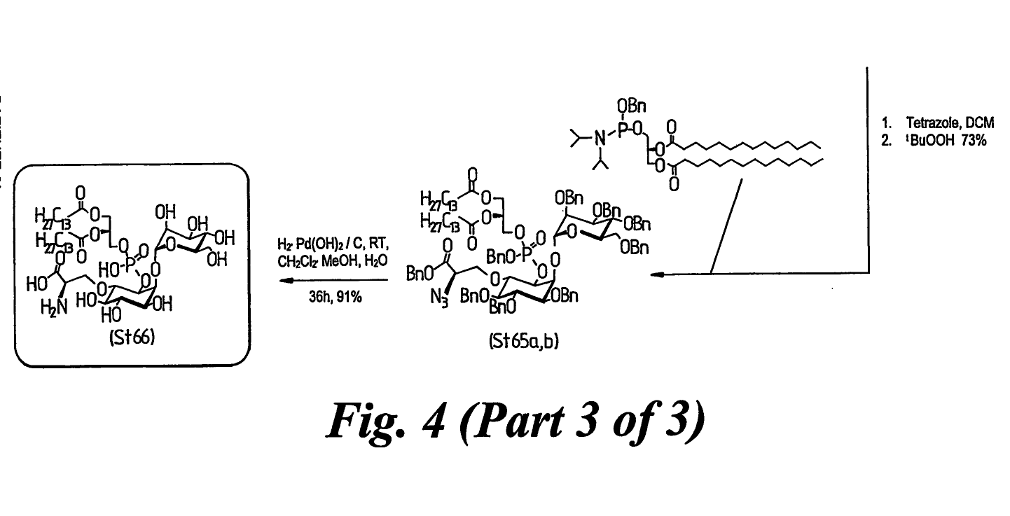

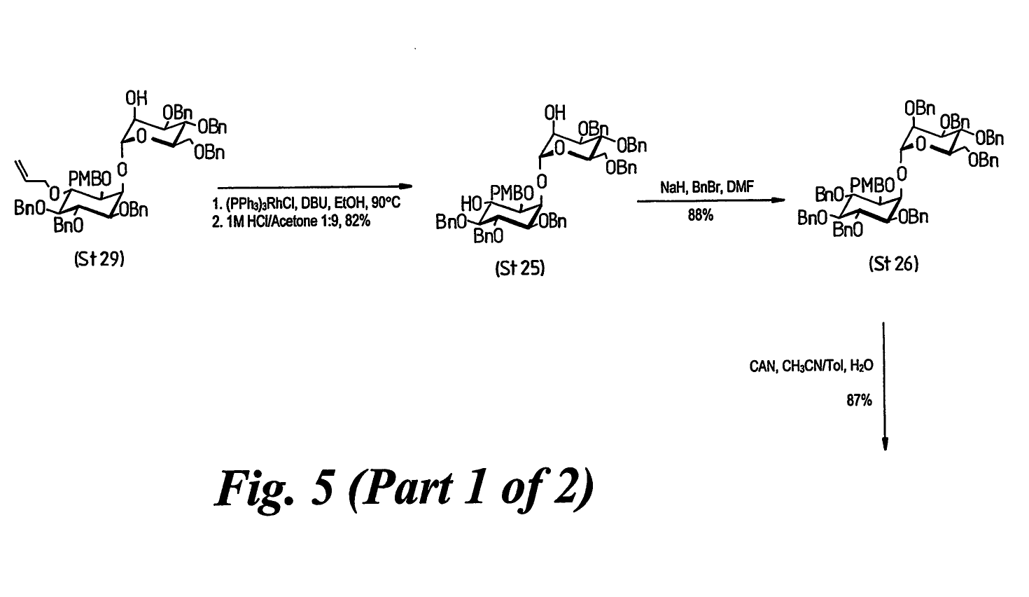

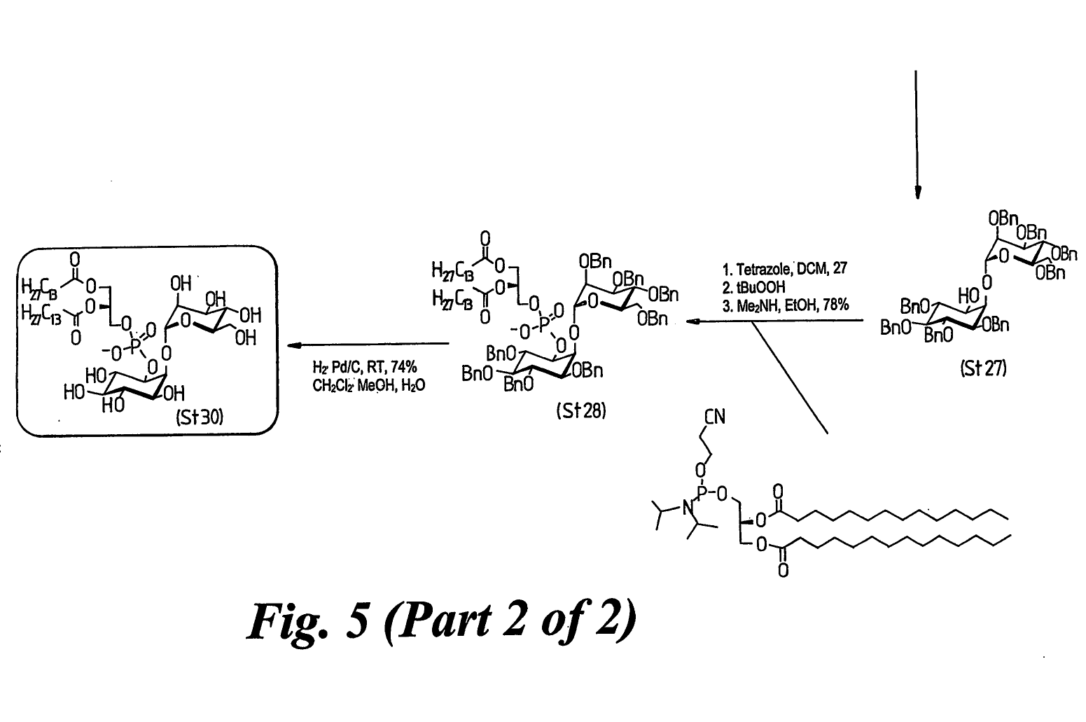

### DETAILED DESCRIPTION

The compounds of the invention may be derivatised in various ways. As used herein “derivatives” of the compounds includes salts, coordination complexes with metal ions such as Mn²⁺ and Zn²⁺, esters such as in vivo hydrolysable esters, free acids or bases, hydrates, prodrugs or lipids, coupling partners.

Salts of the compounds of the invention are preferably physiologically well tolerated and non toxic. Many examples of salts are known to those skilled in the art. Compounds having acidic groups, such as phosphates or sulfates, can form salts with alkaline or alkaline earth metals such as Na, K, Mg and Ca, and with organic amines such as triethylamine and Tris (2-hydroxyethyl)amine. Salts can be formed between compounds with basic groups, e.g. amines, with inorganic acids such as hydrochloric acid, phosphoric acid or sulfuric acid, or organic acids such as acetic acid, citric acid, benzoic acid, fumaric acid, or tartaric acid. Compounds having both acidic and basic groups can form internal salts.

Esters can be formed between hydroxyl or carboxylic acid groups present in the compound and an appropriate carboxylic acid or alcohol reaction partner, using techniques well known in the art.

Derivatives which as prodrugs of the compounds are convertible in vivo or in vitro into one of the active compounds. Typically, at least one of the biological activities of compound will be reduced in the prodrug form of the compound, and can be activated by conversion of the prodrug to release the compound or a metabolite of it.

Other derivatives include coupling partners of the compounds in which the compounds is linked to a coupling partner, e.g. by being chemically coupled to the compound or physically associated with it. Examples of coupling partners include a label or reporter molecule, a supporting substrate, a carrier or transport molecule, an effector, a drug, an antibody or an inhibitor. Coupling partners can be covalently linked to compounds of the invention via an appropriate functional group on the compound such as a hydroxyl group, a carboxyl group or an amino group. Other derivatives include formulating the compounds with liposomes.

The compounds described herein or their derivatives can be formulated in pharmaceutical compositions, and administered to patients in a variety of forms, in particular to treat conditions which are ameliorated by the administration of compound or a coupling partner thereof.

Pharmaceutical compositions for oral administration may be in tablet, capsule, powder or liquid form. A tablet may include a solid carrier such as gelatin or an adjuvant or an inert diluent. Liquid pharmaceutical compositions generally include a liquid carrier such as water, petroleum, animal or vegetable oils, mineral oil or synthetic oil. Physiological saline solution, or glycols such as ethylene glycol, propylene glycol or polyethylene glycol may be included. Such compositions and preparations generally contain at least 0.1 wt % of the compound.

Parenteral administration includes administration by the following routes: intravenous, cutaneous or subcutaneous, nasal, intramuscular, intraocular, transepithelial, intraperitoneal and topical (including dermal, ocular, rectal, nasal, inhalation and aerosol), and rectal systemic routes. For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, solutions of the compounds or a derivative thereof, e.g. in physiological saline, a dispersion prepared with glycerol, liquid polyethylene glycol or oils.

In addition to one or more of the compounds, optionally in combination with other active ingredient, the compositions can comprise one or more of a pharmaceutically acceptable excipient, carrier, buffer, stabiliser, isotonicizing agent, preservative or anti-oxidant or other materials well known to those skilled in the art. Such materials should be non-toxic and should not interfere with the efficacy of the active ingredient. The precise nature of the carrier or other material may depend on the route of administration, e.g. orally or parenterally.

Liquid pharmaceutical compositions are typically formulated to have a pH between about 3.0 and 9.0, more preferably between about 4.5 and 8.5 and still more preferably between about 5.0 and 8.0. The pH of a composition can be maintained by the use of a buffer such as acetate, citrate, phosphate, succinate, Tris or histidine, typically employed in the range from about 1 mM to 50 mM. The pH of compositions can otherwise be adjusted by using physiologically acceptable acids or bases.

Preservatives are generally included in pharmaceutical compositions to retard microbial growth, extending the shelf life of the compositions and allowing multiple use packaging. Examples of preservatives include phenol, meta-cresol, benzyl alcohol, para-hydroxybenzoic acid and its esters, methyl paraben, propyl paraben, benzalconium chloride and benzethonium chloride. Preservatives are typically employed in the range of about 0.1 to 1.0% (w/v).

Preferably, the pharmaceutically compositions are given to an individual in a “prophylactically effective amount” or a “therapeutically effective amount” (as the case may be, although prophylaxis may be considered therapy), this being sufficient to show benefit to the individual. Typically, this will be to cause a therapeutically useful activity providing benefit to the individual. The actual amount of the compounds administered, and rate and time-course of administration, will depend on the nature and severity of the condition being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980. By way of example, and the compositions are preferably administered to patients in dosages of between about 0.01 and 100 mg of active compound per kg of body weight, and more preferably between about 0.5 and 10 mg/kg of body weight.

Experimental

General:

Solvents were purified by distillation and dried as usual, except for distilled CH₂Cl₂ which was passed through a column of commercially available neutral alumina (ICN Alumina N, activity grade super I) as an alternative drying procedure. Boiling range of the petroleum ether: 35-70° C. Thin layer chromatography (TLC) was performed on silica gel Merck Kieselgel 60 F₂₅₄ plates (0.2 mm). The plates were visualized by immersion in mostain [200 ml 10% H₂SO₄, 10 g (NH₄)₆Mo₇O₂₄*4 H₂O, 200 mg Ce(SO₄)₂] or ninhydrin solution (1% in EtOH) or 10% H₂SO₄ or KMnO₄ solution (1% in H₂O, 1% NaHCO₃) followed by heating (165° C.). Preparative flash chromatography was carried out on Baker silica gel 60 (30-60 μm) at a pressure of 0.2-0.4 bar. FAB-MS were recorded on a modified Finnigan MAT 312/AMD 5000. ¹H-NMR, ¹³C-NMR and ³¹P spectra were recorded on a Bruker AC 250 Cryospec and a Bruker DRX 600 instrument. Proton chemical shifts are reported in ppm relative to Me₄Si as internal standard. Assignements of protons and carbons were carried out with the aid of 600 MHz spectra: COSY, HMQC, ROESY, TOCSY. Measurements of optical rotations were performed on a Perkin-Elmer polarimeter 241 MC (1 dm cell). Melting points: Gallenkamp metal block; not corrected. MALDI-MS: Kratos Analytical Kompac Maldi 2; matrix: 2,5-dihydroxybenzoic acid (positive mode), 6-Aza-2-thiothymin (ATT) (negative mode).

Experimental

### 6-O-Allyl-2,3-O-cyclohexyliden-1-O-(1R)-menthyloxycarbonyl-D-myo-inositol (9 D):

Bis-ketal 4 (48.75 g, 0.1 mol) was dissolved in 600 ml of a mixture of dry CH₂Cl₂/MeOH (1:1) and heated up to 40° C.

To this solution was added 10 ml of a mixture of PPTSA:PTSA (13:1, 0.86 M in dry DCM). The reaction was followed by TLC (petrol./EtOAc 1:1, R_{f}=0. 2). Stirring was maintained for 1 h 10 min. before the reaction was quenched with NEt₃. The residue obtained upon evaporation was purified by flash chromatography (petrol ether/EtOAc 3:1→1:1) to yield 9 (28.3 g, 68%) as a colourless foam. TLC petrol ether/EtOAc (1:1). −R_{f}=0, 47−[α]_{D}=−49 (c=1, CHCl₃). −M.p.=129.2° C. −¹H-NMR (250 MHz, CDCl₃) δ 0.75-1.16 (3d, m, 12H, 3Me, 3H_{Mnt}), 1.38-1.79 (14H, 10H_{Cyclohexyliden}, 4H_{Mnt}), 1.9-2.12 (m, 4H, 2H_{Mnt}, 2OH), 3.41 (dd, 1H, J_{5,4=10.3} Hz J_{5,6}=8.6 Hz, 5-Hᵢₙₒₛᵢₜₒₗ), 3.71 (dd, 1H, J_{6,5}=J_{6,1}=8.5 Hz, 6-H_{Inositol}), 3.79 (dd, 1H, J_{4,3}=7.4 Hz, J_{4,5}=10.3 Hz, 4-H_{Inositol}), 4.08 (dd, 1H, J_{3,2}=5.5 Hz, J_{3,4=}7.3 Hz, 3-H_{Inositol}), 4.18-4.38 (m, 2H, OCH₂CH═CH₂), 4.5 (dd, 1H, J_{2,1}=4.1 Hz, J_{2,3}=5.4 Hz, 2-H_{Inositol}), 4.56-4.63 (m, 1H, 1H_{Mnt}),), 4.92 (dd, 1H, J_{1,2}=4.1 Hz, J_{1,6}=8.4 Hz, 1-H_{Inositol}), 5.19-5.36 (m, 2H, CH═CH₂), 5.85-6.01 (m, 1H, CH═CH₂). Anal. Calcd. for C₂₆H₄₂O₈ (482.61) C 64.71 H 8.77; found C 64.69 H 8.60.

### 6-O-Allyl-4,5-di-O-benzyl-2,3-O-cyclohexyliden-1O-(1R)-menthyloxycarbonyl-D-myo-inositol (10 D):

To a solution of 9 (30.89 g, 64 mmol), in anhydrous DMF (400 ml), was treated benzyl bromide (38 ml, 5 equiv.), cooled to 0° C. and added portionwise NaH (3.84 g, 2.5 eqiuv.). The reaction mixture was allowed to reach room temperature and after 2½ h stirring the mixture was quenched with CH₃COOH/EtOAc (1:3) and concentrated in vacuo. The obtained residue was purified on silica gel (petrol ether/ethyl acetate 20:1) to gave 33 g (78%) of product 10 as a colourless syrup. TLC petrolether/EtOAc (6:1)]R_{f}=0.7. −[α]_{D}=−31.9 (c=1, CHCl₃). −¹H-NMR (250 MHz, CDCl₃) δ 0.72-1.13 (3d, m, 12H, 3Me, 3H_{Mnt}), 1.3-1.75 (14H, 10H _{Cyclohexyliden}, 4H_{Mnt}), 1.9-2.12 (m, 2H, 2H_{Mnt}), 3.45 (dd, 1H, J_{5,6}=9.5 Hz, J_{5,4}=8.3 Hz, 5-Hᵢₙₒₛᵢₜₒₗ), 3.79 (dd, 1H, J_{6,5}=9.5 Hz, 6-Hi_{Inositol}), 3.81 (dd, 1H, 4-H_{Inositol}), 4.19-4.23 (m, 3H, CH₂CH═CH₂, 3H_{Inositol}), 4.44 (dd, 1H, J_{2,1}=3.8 Hz, J_{2,3}=5.7 Hz, 2-H_{Inositol}), 4.48-4.6 (m, 1H, 1H_{Mnt}), 4.78 (s, 2H, CH₂Ph), 4.8 (2d, 2H, CH₂Ph), 4.95 (dd, 1H, J_{1,2}=3.8 Hz, J_{1,6}=8.3 Hz, 1-H_{Inositol}), 5.1-5.3 (m, 2H, CH═CH₂), 5.82-5.9 (m, 1H, CH═CH₂), 7.2-7.4 (m, 10H, Ph). −Anal. Calcd. for C₄₀H₅₄O₈ (662,86) C 72.45 H 8.21; found C 72.55 H 8.21. - MALDI-MS: m/z 685.3 [M+Na]⁺, 701.3 [M+K]⁺.

### 6-O-Allyl-4,5-di-O-benzyl-2,3-1-O-(1R)-menthyloxycarbonyl-D-myo-inositol (11 D):

For cleavage of the Ketal, compound 10 (16.74 g, 25 mmol) was dissolved in methanole:dichloromethane (10:1, 220 ml). To this solution was added camphor-10-sulfonic acid (0.75 g, 3.23 mmol) and the reaction mixture was stirred for 8½ h at 45° C. Next the mixture was neutralized with NEt₃, diluted with toluene and concentrated in vacuo. The obtained residue was purified on silica gel (petrol ether/ethyl acetate 6:1) to gave 13.1 g (89% yield) of product 11 as a colourless foam. TLC petrol ether/EtOAc (2:1) R_{f}=0,55. −¹H-NMR(250 MHz, CDCl₃) δ 0.72-1.13 (3d, m, 12H, 3Me, 3H_{Mnt}), 1.32-1.72 (m, 4H, 4H_{Mnt}), 1.88-2.3 (m, 4H, 2H_{Mnt}, 2OH), 3.49 (dd, 1H, 5-Hᵢₙₒₛᵢₜₒₗ), 3.55 (dd, 1H, J_{3,2}=2.7 Hz, J_{3,4}=9.6 Hz, 3-Hᵢₙₒₛᵢₜₒₗ), 3.76 (dd, 1H, 6-H_{Inositol}), 3.89 (dd, 1H, 4-H_{Inositol}), 4.15-4.35 (m, 3H, CH₂CH═CH₂, J_{2,3}=J_{2,1}=2.6 Hz, 2-H_{Inositol}), 4.48-4.6 (m, 1H, 1H_{Mnt}), 4.66-4.97 (4d, 4H, CH₂Ph), 4.7 (dd, 1H, J_{1,2}=2.7 Hz, J_{1,6}=10.1 Hz, 1-H_{Inositol}), 5.08-5.28 (m, 2H, CH═CH₂), 5.8-5.98 (m, 1H, CH═CH₂), 7.21-7.39 (m, 10H, Ph). - Anal. Calcd. for C₃₄H₄₆O₈ (582.73) C 70.08 H 7.96; found C 69.67 H 7.99. - MALDI-MS 605.4 [M+Na]⁺, 621.4 [M+K]⁺.

### 3-O-Acetyl-6-O-allyl-4,5-di-O-benzyl-1-O-(1R)-menthyloxycarbonyl-D-myo-inositol (12 D):

The diol 11 (10.58 g, 18 mmol), dissolved in 100 ml dry THF, was treated with anhydrous K₂CO₃ (5.62 g, 2.24 equiv.) and dimethyltin dichloride (4.39 g, 1.1 equiv.) and stirred for ½ h under argon atmosphere. To this reaction mixture was added acetyl chloride (1.56 ml, 1.2 equiv.) and stirred over night. Then the K₂CO₃was filtered off and the reaction mixture was concentrated in vacuo. Silica gel column chromatography (toluene/EtOAc 15:1) afforded 12 (7.3 g, 65%) as a white solid. TLC toluene/EtOAc (6:1) R_{f}=0.56. −[α]_{D}=−23.5 (c=1, CHCl₃). −M.p.=144.5° C. −¹H-NMR (600 MHz, CDCl₃) δ 0.7-1.08 (3d, m, 12H, 3Me, 3H_{Mnt}), 1.33-1.63 (m, 4H, 4H_{Mnt}), 1.8-1.9, 1.98-2.05 (m, 2H, 2H_{Mnt}), 1.94 (s, 3H, OAc), 3.48 (dd, 1H, J_{5,6}=9.5 Hz, J_{5,4}=9.5 Hz, 5-Hᵢₙₒₛᵢₜₒₗ), 3.82 (dd, 1H, J_{6,1}=J_{6,5}=9.82 Hz, 6-H_{Inositol}), 3.93 (dd, 1H, J4,5=J_{4,3}=9.82 Hz, 4-H_{Inositol}), 4.1-4.16, 4.23-4.27 (m, 2H, CH₂CH═CH₂), 4.19 (dd, 1H, J_{2,3}=J_{2,1}=2.64 Hz, 2-H_{Inositol}) 4.45-4.52 (m, 1H, 1H_{Mnt}), 4.57-4.82 (4d, dd, 5H, CH₂Ph, J_{1,2}=2.64 Hz J_{1,6}=10.2 Hz, 1-H_{Inositol}), 4.85 (dd, 1H, J_{3,4}=10.2 Hz, J_{3,2}=2.83 Hz, 3-H_{Inositol}), 5.03-5.21 (m, 2H, CH═CH₂), 5.8-5.9 (m, 1H, CH═CH₂), 7.14-7.3 (m, 10H, Ph). - Anal. Calcd. for C₃₆H₄₈O₉ (624.77) C 69.21 H 7.74; found C 69.16 H 7.74.

### 3-O-Acetyl-6-O-allyl-4,5-di-O-benzyl-1-O-(1R)-mentyloxycarbonyl-2-O-(2-O-acetyl-3,4,6-tri-O-benzyl-α-D-mannopyranosyl)-D-myo-inositol (28 D):

To a solution of acceptor 12 (8.02 g, 18 mmol), in anhydrous ether (50 ml), was treated under argon atmosphere Sn(OTf)₂ (268 mg, 0.05 equiv.). Then the donor (16.35 g, 0.026 mol, dissolved in 50 ml anhydrous ether) was added dropwise over 5 min. to the reaction mixture. Stirring was maintained for 1 h, then quenched with triethylamine and concentrated. The residue was purified by flash chromatography (toluene/EtOAc 24:1) to gave 28 (13.4 g, 95%) as a colourless foam. TLC toluene/acetone (12:1, 1% NEt₃) R=0.57. −[α]_{D}=+5.9 (c=1, CHCl₃). −¹H-NMR (250 MHz, CDCl₃) δ 0.7-1.11 (3d, m, 12H, 3Me, 3H_{Mnt}), 1.3-1.7 (m, 4H, ⁴H_{Mnt}), 1.78 (s, 3H, OAc), 1.81-2.16 (m, 2H, 2H_{Mnt}), 2.1 (s, 3H, OAc), 3.5-3.6 (m, 2H), 3.66.-3.95 (m, 6H), 4.17-4.37 (m, 3H, OCH₂CH═CH₂), 4.4-4.68 (m, 6H), 4.7-4.9 (m, 7H), 5.0-5.05 (d, 1H, 2-H_{Man}), 5.12-5.31 (m, 2H, CH═CH₂), 5.45 (d, 1H, 1-H_{Man}), 5.8-5.99 (m, 1H, CH═CH₂), 7.1-7.4 (m, 25H, Ph). - Anal. Calcd. for C₆₅H₇₈O₁₅ (1099.32) C 71.02 H 7.15; found C 71.07 H 7.20. - MALDI-MS: 1122.2 [M+Na]⁺.

### 6-O-Allyl-4,5-di-O-benzyl-1-O-(1R)-menthyloxycarbonyl-2-O-(3,4,6-tri-O-benzyl-α-D-mannopyranosyl)-D-myo-inositol (29 D)

Compound 28 (13.4 g, 12 mmol) was dissolved in 100 ml of methylamine solution (33% in anhydrous EtOH) and stirred for 24 h at room temperature. The reaction mixture was concentrated, diluted with toluene and evaporated. Silica gel column chromatography of the residue (toluene/EtOAc 10:1) afforded 29 (9.8 g, 79%) as a colourless foam. TLC petrolether/EtOAc (3:1), R_{f}=0.48. −[α]_{D}=−3.7 (c=1, CHCl₃). −¹H-NMR (250 MHz, CDCl₃) δ 0.72-1.12 (3d, m, 12H, 3Me, 3H_{Mnt}), 1.33-1.72 (m, 4H, 4H_{Mnt}), 1.8-2.2 (m, 4H, 2H_{Mnt}, 2OH), 3.4-3.8 (m, 8H), 4.01-4.12 (m, 2H), 4.12-4.32 (m, 3H, OCH₂CH═CH₂, 2-H_{Inositol}), 4.4-4.89 (m, 12H), 4.96 (d, 1H, 1-H_{Man}), 5.09-5.3 (m, 2H, CH═CH₂), 5.81-5.99 (m, 1H, CH═CH₂), 7.11-7.4 (m, 25H, Ph). −Anal. Calcd. for C₆₁H₇₄O₁₃ (1015.25* ½ H₂O) C 71.53 H 7.38; found C 71.63 H 7.33. - MALDI-MS: 1037.4 [M+Na]⁺, 1053.3 [M+K]⁺.

### 6-O-Allyl-3,4,5-tri-O-benzyl-1-O-(1R)-menthyloxycarbonyl-2-O-(2,3,4,6-tetra-O-benzyl-α-D-mannopyranosyl)-D-myo-inositol (30 D)

To a solution of 29 (9.8 g, 9.65 mmol), in anhydrous DMF (100 ml), was treated benzyl bromide (5.73 ml, 5 equiv.), cooled to 0° C. and added portion wise NaH (579 mg, 2.5 equiv.). The reaction mixture was allowed to reach room temperature and after 2½ h stirring the mixture was quenched with CH₃COOH/EtOAc (1:3) and concentrated in vacuo. The obtained residue was purified on silica gel (petrol ether/ethyl acetate 10:1) to gave 9.75 g (85%) of product 30 as a colourless oil TLC petrol ether/EtOAc (3:1) R_{f}=0.73. −[α]_{D}=−1.8 (c=1, CHCl₃). −¹H-NMR (600 MHz, CDCl₃) δ 0.61-1.08 (3d, m, 12H, 3Me, 3H_{Mnt}), 1.25-1.64 (m, 4H, 4H_{Mnt}), 1.8-1.9, 2.02-2.08 (m, 2H, 2H_{Mnt}), 3.16-3.19, 3.36-3.42 (m, 2H, 6-Hₘₐₙ), 3.28-3.37 (m, 2H, 5-H_{Inositol}, 3-H_{Inositol}), 3.52 (dd, 1H, 6-H_{Inositol}), 3.63 (dd, 1H, 4-H_{Inositol}), 3.64-3.73 (m, 2H, 2-H_{Man}, 3-H_{Man}), 3.9-4.05 (m, 2H, 4-H_{Man}, 5-H_{Man}), 4.08-4.2 (m, 2H, CH₂CH═CH₂), 4.2-4.26, 4.33-4.4, 4.45-4.54, 4.66-4.85 (m, 15H, CH₂Ph, 1-H_{Mnt}), 4.32 (dd, 1H, 2-H_{Inositol}), 4.6 (dd, 1H, 1-H_{Inositol}) 4.97-5.04, 5.1-5.2 (m, 2H, CH=CH₂), 5.27 (d, 1H, 1-H_{Man}), 5.79-5.9 (m, 1H, CH═CH₂), 6.98-7.48 (m, 35H, Ph). - Anal. Calcd. for C₇₅H₈₆O₁₃ (1195.5) C 75.35 H 7.25; found C 75.08 H 7.49. - MALDI-MS 1217.4 [M+Na]⁺.

### 6-O-Allyl-3,4,5-tri-O-benzyl-2-O-(2,3,4,6-tetra-O-benzyl-α-D-mannopyranosyl)-D-myo-inositol (31 D)

To a solution of 30 (3.1 g, 2.6 mmol), in a mixture of 110 ml anhydrous MeOH/ether (10:1), was added K₂CO₃ and stirred for 6 h at 60° C. After evaporation, the residue was purified by silica gel column chromatography (toluene/EtOAc 6:1) to afford 31 (2 g, 86%) as a colourless oil. TLC petrol ether/EtOAc (3:1) R_{f}=0.17. [α]_{D}=+35.1 (c=1, CHCl₃). −¹H-NMR (250 MHz, CDCl₃) δ 3.28-3.42 (m, 5H), 3.52-3.6 (m, 1H), 3.68-3.9 (m, 3H), 3.98-4.15 (m, 3H), 4.31-4.98 (m, 16H), 5.1-5.29 (m, 2H, CH═CH₂), 5.46 (d, 1H, 1-H_{Man}), 5.79-5.96 (m, 1H, CH═CH₂), 7.11-7.4 (m, 35H, Ph). - Anal. Calcd. for C₆₄H₆₈O₁₁ (1013.24) C 75.87 H 6.77; found C 75.96 H 7.02. - MALDI-MS 1035.2 [M+Na]⁺, 1051.2 [M+K]⁺.

### 6-O-Allyl-3,4,5-tri-O-benzyl-2-O-(2,3,4,6-tetra-O-benzyl-α-D-mannopyranosyl)-D-myo-inosit-1-yl-[(2R)-2,3-bis-(myristoyloxy)-propyl]-phosphate (32 D)

Tetrazole (360 mg, 2.6 equiv.) and cyanethoxy-N,N-diisopropylphosphoramidite (2.82 g, 2 equiv.) were dried for 1 h under high vacuum. Compound 31 (2 g, 1.97 mmol, dissolved in 100 ml anhydrous dichloromethane) was added to the mixture of tetrazole and cyanethoxy-N,N-diisopropylphosphoramidite. Stirring was maintained at rt under argon atmosphere for 2 h. The two diastereomers (petrol ether/EtOAc 3:1, R_{f}=0.5 and 0.56) were then treated with tertbutylperoxide (4.7 M in isooctane, 7.85 ml). After 1 h, the reaction mixture was concentrated to 10 ml, treated with dimethylamine solution (36 ml, 33% in anhydrous EtOH) and stirred for 1 h. Then the reaction mixture was again concentrated to 10 ml, diluted with CH₂Cl₂, saturated NaHCO₃-solution was added and the two layers were separated. The organic layer was washed with brine, dried (Na₂SO₄) and concentrated. The residue was purified by flash chromatography (toluene/acetone 9:1→1:10) to afford 32 (2.6 g, 78%) as a slightly yellow oil. −³¹P-NMR [600 MHz, CD₃OD/CDCl₃ (1:1)] δ −3.972 (s, 1P).TLC CHCl₃/MeOH (10:1). −R_{f}=0.34. −¹H-NMR (600 MHz, CDCl₃/MeOH 1:1) δ 0.8-0.95 (t, 6H, Me), 1.04-1.4 (s, 40H, CH₂-Kette), 1.4-1.7 (m, 4H, COCH₂—CH₂—R), 2.18-2.3 (m, 4H, COCH₂—CH₂—R), 3.1 (m, 6-H_{Man}), 3.29-3.43 (m, 3H, 6-H_{Man,}5-H_{Inositol}, 3-H_{Inositol}), 3.59 (dd, 1H, 6-H_{Inositol}), 3.72 (dd, 1H, 4-H_{Inositol}), 3.79 (dd, 1H, 2-H_{Man}), 3.82 (dd, 1H, 3-H_{Man}), 4.0-4.18 (m, 4,5H, 4-H_{Man}, 1-H_{Inositol}, 1″, 3″), 4.25-4.4 (m, 2H, OCH₂CH═CH₂), 4.2-4.25, 4.4-4.9 (m, 14H, CH₂Ph), 4.58 (dd, 1H, 2-H_{Inositol}) t 5.13-5.32 (m, 3H, CH═CH₂, 2″), 5.61 (d, 1H, 1-H_{Man}), 5.9-6.1 (m, 1H, CH═CH₂), 7.1-7.52 (m, 35H, Ph). −C₉₇H₁₃₄O₁₈NP (1633.1) (dimethylammonium salt); C₉₅H₁₂₇O₁₈P (1587.09) (free acid). - MALDI-MS: 1587,6 [M−H)⁻.

### 3,4,5-Tri-O-benzyl-2-O-(2,3,4,6-tetra-O-benzyl-α-D-mannopyranosyl)-D-myo-inosit-1-yl-[(2R)-2,3-bis-[(myristoyloxy)-propyl]-phosphate (35 D)

To a solution of 32 (310 mg, 0.2 mmol), in CH₂Cl₂ (2 ml), was added under argon atmosphere Pd(PPh₃)₄ (22 mg, 0.1 equiv.), paratoluenesulfinic acid sodium salt (40.6 mg, 1.2 equiv.) and acetic acid (26.1 μl, 2.4 equiv.). The reaction mixture was stirred for 2 h at room temperature, concentrated in vacuo and purified by silica gel column chromatography CHCl₃→CHCl₃/MeOH 48:1) to yield 35 (180 mg, 58%) as a colourless oil. TLC CHCl₃/MeOH (4:1) R_{f}=0.69. −¹H-NMR (600 MHz, CDCl₃/MeOH 1:1) δ 0.82-0.95 (t, 6H, Me), 1.17-1.4 (s, 40H, CH₂-Kette), 1.45-1.65 (m, 4H, COCH₂—CH₂—R), 2.2-2.3 (m, 4H, COCH₂—CH₂—R), 3.15-3.25 (m, 1H), 3.3-3.5 (m, 3H), 3.73-4.3 (m, 11H), 4.39-4.98 (m, 16H), 5.22-5.35 (m, 1H, 2″), 5.53 (d, 1H, 1-H_{Man}), 7.1-7.5 (m, 35H, Ph). —C₉₂H₁₂₃O₁₈P (1547.06) (free acid).

### Triethylammonium-[2-O-(α-D-mannopyranosyl)-D-myo-inosit-1-yl]-[(2R)-2,3-bis-(myristoyloxy)-propyl]-phosphate (36 D)

A vigorously stirred mixture of 35 (170 mg, 0.1 mmol, CH₂Cl₂:MeOH:H₂O (7.5:7.5:1, 3 ml)) and Pd/C (0.2 equiv.) was degassed under vacuum and saturated with hydrogen (by a H₂-filled balloon) three times. The suspension was stirred at room temperature over night, filtered over celite, washed with CH₂Cl₂:MeOH:H₂O (7.5:7.5:1, 2 ml) and treated with some triethylamine. The solvents were removed under vacuum by lyophilisation to afford 36 (82 mg, 73%) as a white solid. R_{f} (CHCl₃/MeOH/0.2% CaCl₂-solution 65:35:8)=0.26. −³¹P-NMR (600 MHz, dmso) δ 1.305 (s, 1P). −¹H-NMR (600 MHz, dmso) δ 0.75-0.91 (t, 6H, Me), 1.05-1.37 (m, 49H, CH₂-Chain, Me_{NEt3}), 1.39-1.57 (m, 4H, COCH₂CH₂R), 2.17-2.35 (m, 4H, COCH₂CH₂R), 2.93 (m, 1H, 5-H_{Inositol}), 3.08 (m, 6H, HN(CH₂—CH₃)₃), 3.18 (m, 1H, 3-H_{Man}), 3.32 (m, 1H, 4-H_{Inositol}), 3.47 (m, 2H, 6-H_{Man}, 6-H_{Inositol}), 3.48 (m, 1H, 4-H_{Man}), 3.51 (m, 1H, 3-H_{Man}), 3.54 (m, 1H, 6-H_{Man}), 3.64 (m, 1H, 2-H_{Man}), 3.72 (m, 1H, 1-H_{Inositol}), 3.77 (m, 1H, 1′/3′), 3.84 (m, 1H, 5-H_{Man}), 3.89 (m, 1H, 1′/3′), 4.00 (m, 1H, 2-H_{Inositol}) 4.1 (m, 1H, 1′/3′), 4.31 (m, 1H, 1′/3′), 5.02 (m, 1H, 1-H_{Man}), 5.09 (m, 1H, 2′). - MALDI: calcd. (M−H⁺)⁻ m/z=915.67; found m/z=914.9.C₄₉H₉₆O₁₈NP (1017.8) (triethylammonium salt); C₄₃H₈₀O₁₈P (916.67) (free acid).

4a: Separation procedure: The mother liquors were evaporated and flash chromatography (petrol ether/EtOAc 8:1, 11 g was dissolved in CH₂Cl₂, treated with 100 g silica gel and evaporated to dryness. Column size: Ø=7 cm, 1=40 cm) to yield pure 4a (8.6 g). TLC (petrol ether/EtOAc 3:1), R_{f}=0.44.

4b: TLC (petrol ether/EtOAc 3:1), R_{f}=0.37.

### St 6: (−)-6-O-Allyl-3-O-benzyl-1,2:5,6-di-O-cyclohexyliden-D-myo-inositol

A solution of 5b (24.95 g, 58 mmol), in dry DMF (620 ml), was treated with AllBr (6.38 ml, 75.43 mmol), then sodium hydride (2.5 g, 0.1 mol) was added. The reaction mixture was stirred at rt. for 45 minutes, quenched with MeOH and concentrated in vacuo. The residue was diluted with EtOAc, washed with water and brine. The organic layer was dried with MgSO₄ and concentrated under reduced pressure. The resulting syrup was applied to a short column of silica gel which was eluted with petroleum/ethyl acetate (9:1→3:1) to gave 6 as a white solid (25.1 g, 92%). TLC (petrol ether/EtOAc 3:1), R_{f}=0.67. −M.p.: 98-99° C. −[α]_{D}=−41.5 (c=1, CHCl₃). −¹H-NMR (600 MHz, CDCl₃) δ 1.3-1.85 (m, 20H, H_{Cyclo}.), 3.27 (dd, 1H, J_{5,6}=J_{5,4} =10 Hz, 5-H_{Inositol}), 3.63 (dd, 1H, J_{6,1} =6.46 Hz, J_{6,5}=10.56 Hz, 6-H_{Inositol}), 3.73 (dd, 1H, J_{3,4}=10,27 Hz, J_{3,2}=4.11 Hz, 3-H_{Inositol}), 4.00 (dd, 1H, J_{1,2}=4.99 Hz, J_{1,6}=6.75 Hz, 1-H_{Inositol}), 4.03 (dd, 1H, J_{4,5}=J_{4,3}=9.68 Hz, 4-H_{Inositol}), 4.25-4.31 (m, 2H, CH₂CH═CH₂), 4.33 (dd, 1H, J_{2,3}=J_{2,1}=4.40 Hz, 2-H_{Inositol}), 4.80-4.91 (m, 2H, CH₂-Ph), 5.14-5.21; 5.3-5.35 (m, 2H, CH₂CH═CH₂), 5.90-5.99 (m, 1H, 2H, CH₂CH═CH₂), 7.24-7.44 (m, 5H, ArH).). —¹³C-NMR (150.9 MHz, CDCl₃) δ 23.56, 23.83, 23.86, 23.93, 24.98, 25,07, 35.25, 36.38, 36.49, 37.61 (10C, C_{cyclohexan}), 71.2 (1C, CH₂—═CH₂), 71.61 (1C, CH₂Ph), 74.58 (1C, 3-C), 76.26 (1C, 4-C), 76.68 (1C, 2-C), 78.42 (1C, 1-C), 80.36 (1C, 6-C), 80.9 (1C, 5-C), 110.44, 112.63 (2C, C_{cyclohexan}), 117.1 (1C, CH₂—═CH₂), 134.92 (1C, CH₂—═CH₂), 127.72-138.12 (6C, Ph). - Anal. calcd. for C₂₈H₃₈O₆ (470.61): C 71.46, H 8.14; found: C 71.26, H 7.97. - MALDI: calcd. M+Sodium, m/z=493.6; found m/z=494.3.

### St 13: (+)-6-O-Allyl-3-O-benzyl-1,2:5,6-di-O-cyclohexyliden-L-myo-inositol

Repetition of the above procedure but starting with 5a afforded the title compound 13.

TLC (petrol/EtOAc 3:1), R_{f}=0.67. −M.p.: 90.2° C. −[α]_{D=}+41.7 (c=1, CHCl₃). −¹H-NMR (600 MHz, CDCl₃) δ 1.3-1.85 (m, 20H, H_{Cyclo}.), 3.27 (dd, 1H, J_{5,6}=J_{5,4}=10 Hz, 5-H_{Inositol}), 3.63 (dd, 1H, J_{6,1}=6.46 Hz, J_{6,5}=10.56 Hz, 6-H_{Inositol}), 3.73 (dd, 1H, J_{3,4}=10.27 Hz, J_{3,2}=4.11 Hz, 3-H_{Inositol}), 4.00 (dd, 1H, J_{1,2}=4.99 Hz, J_{1,6}=6.75 Hz, 1-H_{Inositol}), 4.03 (dd, 1H, J_{4,5}=J_{4,3}=9. 68 Hz, 4-H_{Inositol}), 4.25-4.31 (m, 2H, CH₂CH═CH₂), 4.33 (dd, 1H, J_{2,3}=J_{2,1}=4.40 Hz, 2-H_{Inositol}), 4.80-4.91 (m, 2H, CH₂-Ph), 5.14-5.21, 5.3-5.35 (m, 2H, CH₂CH═CH₂), 5.90-5.99 (m, 1H, 2H, CH₂CH═CH₂), 7.24-7.44 (m, 5H, ArH). - Anal. calcd. for C₂₈H₃₈O₆ (470.61): C 71.46, H 8.14; found C 71.43, H 8.14.

### St 7: (+)-6-O-Allyl-3-O-benzyl-1,2-O-cyclohexyliden-D-myo-inositol

Bis-ketal 6 (10 g, 21.28 mmol) was dissolved in 200 ml of a mixture of dry CH₂Cl₂/MeOH (1:1) and heated up to 40° C. To this solution was added 0.3 ml of a mixture of PPTSA:PTSA (13:1, 0.86 M in dry DCM). The reaction was followed by TLC (petrol./EtOAc 1:1, R_{f}=0.2). Stirring was maintained for 1 h 10 min. before the reaction was quenched with NEt₃. The residue obtained upon evaporation was purified by flash chromatography (petrol ether/EtOAc 1:1, dissolved in minimum of EtOAc/MeOH) to yield 7 (6.4 g, 77.5%). M.p.: 135° C. −[α]_{D}=+15.8 (c=1, CHCl₃). −¹H-NMR (600 MHz, CDCl₃) δ 1.3-1.8 (m, 10H, H_{Cyclo}.), 2.73 (s, 2H, OH), 3.33 (dd, 1H, J_{5,6}=J_{5,4}=9. 68 Hz, 5-H_{Inositol}), 3.46 (dd, 1H, J_{4,5}=9.68, J_{4,3}=7.04 Hz, 4-H_{Inositol}), 3.52 (dd, 1H, J_{1,2}=4.11 Hz, J_{1,6}=9. 68 Hz, 1-H_{Inositol}), 3.94 (dd, 1H, J_{6,1}=J_{6,5}=9.39 Hz, 6-Hᵢₙₒₛᵢₜₒₗ), 4.01 (dd, 1H, J_{3,4}=7.04 Hz, J_{3,2}=4.99 Hz, 3-H_{Inositol}), 4.18-4.21, 4.39-4.42 (m, 2H, CH₂CH═CH₂), 4.31 (dd, 1H, J_{2,3}=J_{2,1}=4.7 Hz, 2-H_{Inositol}), 4.73-4.79 (m, 2H, CH₂-Ph), 5.17-5.20, 5.27-5.30 (m, 2H, CH₂CH═CH₂), 5.91-5.98 (m, 1H, 2H, CH₂CH═CH₂), 7.25-7.44 (m, 5H, ArH). —¹³C-NMR (150.9 MHz, CDCl₃) δ 23.66, 23.92, 24.98, 35.17, 37.76 (5C, C_{cyclohexan}), 71.47 (1C, 6-C), 72.31, 72.35 (2C, CH₂—═CH₂, CH₂Ph), 72.85 (1C, 5-C), 73.37 (1C, 2-C), 77.27 (1C, 1-C), 78.87 (1C, 3-C), 81.98 (1C, 4-C), 110.57 (1C, C_{cyclohexan}), 117.35 (1C, CH₂—═CH₂), 128.02-137.89 (6C, Ph), 134.86 (1C, CH₂—═CH₂). - Anal. calcd. for C₂₂H₃₀O₆ (390.48): C 67.67, H 7.74; found C: 67.52, H 7.81. - MALDI: calcd. M+Sodium m/z=413.48; found: m/z=413.4.

### St 14: (−)-6-O-Allyl-3-O-benzyl-1,2-O-cyclohexyliden-L-myo-inositol

Repetition of the above procedure but starting with 13 afforded the title compound 14.

M.p.: 138° C. −[α]_{D}=−18.1 (c=1, CHCl₃). —¹H-NMR (600 MHz, CDCl₃) δ 1.3-1.8 (m, 10H, H_{Cyclo}), 2.73 (s, 2H, OH), 3.33 (dd, 1H, J_{5,6}=J_{5,4}=9.68 Hz, 5-H_{Inositol}), 3.46 (dd, 1H, J_{4,5}=9.68, J_{4,3}=7.04 Hz, 4-H_{Inositol}), 3.52 (dd, 1H, J_{1,2}=4.11 Hz, J_{1,6}=9.68 Hz, 1-H_{Inositol}), 3.94 (dd, 1H, J_{6,1}=J_{6,5}=9.39 Hz, 6-H_{Inositol}), 4.01 (dd, 1H, J_{3,4}=7.04 Hz, J_{3,2}=4.99 Hz, 3-H_{Inositol}), 4.18-4.21, 4.39-4.42 (m, 2H, CH₂CH═CH₂), 4.31 (dd, 1H, J_{2,3}=J_{2,1}=4.7 Hz, 2-H_{Inositol}), 4.73-4.79 (m, 2H, CH₂-Ph), 5.17-5.20, 5.27-5.30 (m, 2H, CH₂CH═CH₂), 5.91-5.98 (m, 1H, 2H, CH₂CH═CH₂), 7.25-7.44 (m, 5H, ArH). - Anal. calcd. for C₂₂H₃₀O₆ (390.48): C 67.67, H 7.74; found C 67.48, H 7.61.

### St 8: (−)-6-O-Allyl-3,4,5-tri-O-benzyl-1,2-O-cyclohexyliden-D-myo-inositol

To a solution of 7 (10.92 g, 28 mmol), in dry DMF (300 ml), was added BnBr (9 ml, 75.6 mmol). NaH (1.68 g, 70 mmol) was slowly added to the solution and after stirring for 3 h at room temperature, the reaction mixture was treated with MeOH. After usual workup (EtOAC/water) the organic layer was dried (MgSO₄) and concentrated in vacuo. Flash chromatography (petrol ether/EtOAc 10:1) gave 8 (13.1 g, 82%) as a colourless syrup. TLC (petrol ether/EtOAc 8:1), R_{f}=0.35. −[α]_{D}=−41 (c=1, CHCl₃). —¹H-NMR (250 MHz, CDCl₃) 6 1.3-1.88 (m, 10H, H_{Cyclo}.), 3.35 (dd, 1H, J=8.62, J=9.76 Hz, H_{Inositol}), 3.63-3.75 (m, 2H, H_{Inositol}), 3.91 (dd, 1H, J=8.64 Hz, H_{Inositol}), 4.0 (dd, 1H, J=5.5 Hz, J=7.06 Hz, H_{Inositol}) 4.2-4.31, 4.32-4.43 (m, 3H, 2-H_{Inositol}, CH₂CH═CH₂), 4.70-4.89 (m, 6H, CH₂-Ph), 5.12-5.36 (m, 2H, CH₂CH═CH₂), 5.89-6.08 (m, 1H, CH₂CH═CH₂), 7.20-7.47 (m, 15H, ArH). - Anal. calcd. for C₃₆H₄₂O₆ (570.73): C 75.76, H 7.42; found C 75.74, H 7.41. - MALDI: calcd. M+Sodium m/z=593.73; found m/z=591.5.

### St 16: (+)-6-O-Allyl-3,4,5-tri-O-benzyl-1,2-O-cyclohexyliden-L-myo-inositol

Repetition of the above procedure but starting with 14 afforded the title compound 16.

[α]_{D}=+43.1 (c=1, CHCl₃). —¹H-NMR (250 MHz, CDCl₃) δ 1.3-1.88 (m, 10H, H_{Cyclo}.), 3.35 (dd, 1H, J=8.62, J=9.76 Hz, H_{Inositol}), 3.63-3.75 (m, 2H, H_{Inositol}), 3.91 (dd, 1H, J=8.64 Hz, H_{Inositol}), 4.0 (dd, 1H, J=5.5 Hz, J=7.06 Hz, H_{Inositol}), 4.2-4.31, 4.32-4.43 (m, 3H, 2-H_{Inositol}, CH₂CH═CH₂), 4.70-4.89 (m, 6H, CH₂-Ph), 5.12-5.36 (m, 2H, CH₂CH═CH₂), 5.89-6.08 (m, 1H, CH₂CH═CH₂), 7.20-7.47 (m, 15H, ArH). - Anal. calcd. for C₃₆H₄₂O₆ (570.73): C 75.76, H 7.42; found C 75.78, H 7.40.

### St 9: (−)-6-O-Allyl-3,4,5-tri-O-benzyl-D-myo-inositol

For cleavage of the Ketal, compound 8 (14.51 g, 25 mmol) was dissolved in methanole:dichloromethane (10:1, 220 ml). To this solution was added camphor-10-sulfonic acid (0.75 g, 3.23 mmol) and the reaction mixture was stirred for 24 h at room temperature. Next the mixture was neutralized with NEt₃, diluted with toluene and concentrated in vacuo. The obtained residue was purified on silica gel (petrol ether/ethyl acetate 1:1) to gave 11 g (88% yield) of product 9 as a white solid. TLC: (petrol ether/EtOAc 2:1), R_{f}=0.25. −M.p.: 123° C. −[α]_{D}=−34.2 (c=1, CHCl₃). —¹H-NMR (600 MHz, CDCl₃) δ 2.5 (s, 2H, OH), 3.41 (dd, 1H, J_{5,6}=J_{5,4}=9.4 Hz, 5H_{Inositol}), 3.44-3.47 (m, 2H, 1-H_{Inositol}, 3-H_{Inositol}), 3.70 (dd, 1H, J_{6,1}=J_{6,5}=9.4 Hz, 6-H_{Inositol}), 3.92 (dd, 1H, J_{4,5}=J_{4,3}=9.4 Hz, 4-H_{Inositol}), 4.21 (dd, 1H, J_{2,3}=J_{2,1}=3.0 Hz, 2-H_{Inositol}), 4.22-4.28, 4.39-4.44 (m, 2H, CH₂CH═CH₂), 4.68-4.70, 4.76-4.89 (m, 6H, CH₂-Ph), 5.16-5.18; 5.25-5.29 (m, 2H, CH₂CH═CH₂), 5.92-5.98 (m, 1H, 2H, CH₂CH═CH₂), 7.25-7.34 (m, 15H, ArH). —¹³C-NMR (150.9 MHz, CDCl₃) δ 69.20 (1C, 2-C), 71.70 (1C, 1-C), 72.78 (1-C, CH₂Ph), 74.35 (1C, CH₂—═CH₂), 75.67 (1-C, CH₂Ph), 75.92 (1-C, CH₂Ph), 80.01 (1C, 3-C), 80.90 (1C, 6-C), 81.60 (1C, 4-C), 83.17 (1C, 5-C), 117.35 (1C, CH₂—═CH₂), 127.62-138.66 (18C, Ph), 134.97 (1C, CH₂—═CH₂). - Anal. calcd. for C₃₀H₃₄O₆ (490.60): C 73.45, H 6.99; found C 73.54, H 7.04.

### St 18: (+) -6-O-Allyl-3,4,5-tri-O-benzyl-L-myo-inositol

Repetition of the above procedure but starting with 16 afforded the title compound 18.

M.p.: 123° C. −[α]_{D}=+31 (c=1, CHCl₃). —¹H-NMR (600 MHz, CDCl₃) δ 2.5 (s, 2H, OH), 3.41 (dd, 1H, J_{5,6}=J_{5,4}=9.4 Hz, 5-H_{Inositol}), 3.44-3.47 (m, 2H, 1-H_{Inositol}, 3-H_{Inositol}), 3.70 (dd, 1H, J_{6,1}=J_{6,5}=9.4 Hz, 6-H_{Inositol}) 3.92 (dd, 1H, J_{4,5}=J_{4,3}=9.4 Hz, 4-H_{Inositol}), 4.21 (dd, 1H, J_{2,3}=J_{2,1}=3.0 Hz, 2-H_{Inositol}), 4.22-4.28, 4.39-4.44 (m, 2H, CH₂CH═CH₂), 4.68-4.70, 4.76-4.89 (m, 6H, CH₂-Ph), 5.16-5.18; 5.25-5.29 (m, 2H, CH₂CH═CH₂), 5.92-5.98 (m, 1H, 2H, CH₂CH═CH₂), 7.25-7.34 (m, 15H, ArH). - Anal. calcd. for C₃₀H₃₄O₆ (490.60): C 73.45, H 6.99; found C 73.5, H 6.87.

### St 33: (−)-6-O-Allyl-3,4,5-tri-O-benzyl-1-O-(4-methoxybenzyl)-D-myo-inositol

Repetition of the above procedure but starting with 9 afforded the title compound 33.

M.p.: 105° C. −[α]_{D}=−10 (c=0.9, CDCl₃). —¹H-NMR (250 MHz, CDCl₃) δ 2.41 (s, 1H, OH), 3.29 (dd, 1H, J=9.6 Hz, J=2.7 Hz, H_{Inositol}), 3.36 (dd, 1H, J=9.7 Hz, J=2.8 Hz, H_{Inositol}), 3.38 (dd, 1H, J=9.5 Hz, H_{Inositol}), 3.81 (s, 3H, OMe), 3. 81 (dd, 1H, J=9.5 Hz, H_{Inositol}), 3. 94 (dd, 1H, J=9.6 Hz, H_{Inositol}), 4.16 (dd, 1H, J=2.7 Hz, H_{Inositol}), 4.28, 4.95 (m, 2H, CH₂CH═CH₂), 4.59-4.74, 4.78-4.93 (m, 8H, CH₂-Ph), 5.12-5.34 (m, 2H, CH₂CH═CH₂), 5.90-6.08 (m, 1H, 2H, CH₂CH═CH₂), 6.83-6.92 (m, 2H, H_{PMB}), 7.22-7.40 (m, 17H, ArH). - Anal. calcd. for C₃₈H₄₂O₇*¼ H₂O (615.25): C 71.18, H 6.96; found C 73.97, H 6.71.

### St 20: (+)-6-O-Allyl-3,4,5-tri-O-benzyl-1-O-(4-methoxybenzyl)-L-myo-inositol

Compound 18 (15.1 g, 30.77 mmol), in anhydrous toluene (500 ml), was treated with dibutyltinoxide (8.43 g, 1.1 eqiv.) and the reaction mixture was refluxed in an apparatus for the azeotropic removal of water for 3 h. To the reaction mixture was added TBAI (17 g, 46 mmol) and para methoxybenzyl chloride (9.1 ml, 67 mmol). Stirring was maintained at 110° C. for 1.5 h, then evaporated and diluted with ethylacetate, washed with water, brine, dried (MgSO₄) and concentrated. Silica gel column chromatography of the residue (petrol ether/EtOAc 6:1→5:1) yielded 20 (14.85 g, 79%) as a slightly brown solid. TLC: (petrol ether/EtOAc 2:1), R_{f}=0.43. −M.p.: 106° C. −[α]_{D}=+9.1 (c=1, CHCl₃). —¹H-NMR (250 MHz, CDCl₃) δ 2.41 (s, 1H, OH), 3.29 (dd, 1H, J=9.6 Hz, J=2.7 Hz, H_{Inositol}), 3.36 (dd, 1H, J=9.7 Hz, J=2.8 Hz, H_{Inositol}), 3.38 (dd, 1H, J=9.5 Hz, H_{Inositol}), 3.81 (s, 3H, OMe), 3.81 (dd, 1H, J=9.5 Hz, H_{Inositol}), 3.94 (dd, 1H, J=9.6 Hz, H_{Inositol}), 4.16 (dd, 1H, J=2.7 Hz, H_{Inositol}),4.28, 4.95 (m, 2H, CH₂CH═CH₂), 4.59-4.74, 4.78-4.93 (m, 8H, CH₂-Ph), 5.12-5.34 (m, 2H, CH₂CH═CH₂), 5.90-6.08 (m, 1H, 2H, CH₂CH═CH₂), 6.83-6.92 (m, 2H, H_{PMB}), 7.22-7.40 (m, 17H, ArH). - Anal. calcd. for C₃₈H₄₂O₇ (610.75): C 73.64, H 6.99; found C 73.70, H 6.74.

### St 34: (+)-6-O-Allyl-3,4,5-tri-O-benzyl-1-O-(4-methoxybenzyl)-2-O-(2-O-acetyl-3,4,6-tri-O-benzyl-α-D-mannopyranosyl)-(1→2)-D-myo-inositol

A mixture of imidate (15 g, 23.55 mmol) and acceptor 33 (11 g, 18 mmol) were dissolved in anhydrous ether (200 ml). Trimethylsilyltriflate (0.5 ml, 2.77 mmol) was added and the mixture was stirred for ten seconds, then quenched with triethylamine, diluted with toluene and concentrated. The residue was purified by flash chromatography (petrol ether/EtOAc 5:1→4:1) to gave 34 (14 g, 72%). TLC: (petrol ether/EtOAc 2:1, 1% NEt₃), R_{f}=0.65. −[α]_{D}=+20.1 (c=1, CHCl₃). —¹H-NMR (600 MHz, CDCl₃) δ 2.09 (s, 3H, OAc), 3.24-3.28 (m, 3H, 6-H_{Man}, 1-H_{Inositol}, 3-H_{Inositol}), 3.37 (dd, 1H, J_{5,6}=J_{5,4}=9.5 Hz, 5-H_{Inositol}), 3.49 (dd, 1H, J_{vic.}=7.37 Hz, J_{gem.}=10.75 Hz, H_{Man}), 3.75-3.8 (m, 5H, OMe, 6-H_{Inositol}, 4-H_{Inositol}), 3.9-3.98 (m, 2H, 3-H_{Man}, 4-H_{Man}), 4.14 (m, 1H, 5-H_{Man}), 4.27 (dd, 1H, J_{2,3}=J_{2,1}=2.38 Hz, 2-H_{Inositol}), 4.28-4.41, 4.5-4.87 (m, 16H, CH₂CH═CH₂, CH₂-Ph), 5.15-5.18; 5.27-5.3 (m, 2H, CH₂CH═CH₂), 5.25 (s, 1H, 1-H_{Man}), 5.48 (dd, 1H, J_{2,3}=J_{2,1}=2.32 Hz, ²-H_{Man}), 5.92-6.01 (m, 1H, 2H, CH₂CH═CH₂), 6.83-6.86 (m, 2H, H_{PMB}), 7.05-7.37 (m, 32H, ArH). - ¹³C-NMR (150.9 MHz, CDCl₃) δ 21.14 (1C, COCH₃), 55.25 (1C, OMe), 68.46 (1C, 6-C_{Man}), 68.63 (1C, 2-C_{Man}), 71.32 (1C, 5C_{Man}), 71.78-76.16 (10C, CH₂Ph, 2-C_{Inositol}, CH₂—═CH₂, 4-C_{Man}), 77.78 (1C, 3-C_{Man}), 78.8 (1C, 1-C_{Inositol}/3C_{Inositol}), 80.04 (1C, 1-C_{Inositol}/3-C_{Inositol}), 81.03 (1C, 4C_{Inositol}/6C_{Inositol}), 81.13 (1C, 4-C_{Inositol}/6-C_{Inositol}), 83.41 (1C, 5-C_{Inositol}), 98.91 (1C, 1-C_{Man}), 113.74 (2C, C_{PMB}), 116.83 (1C, CH₂-CH═CH₂), 127.24-128.24, 137.98-138.71 (39C, Ph), 135.35 (1C, CH₂—═CH₂), 159.18 (1C, C_{OMe}), 171.0 (1C, COCH₃). —Anal. calcd. for C₆₇H₇₂O₁₃ (1085.3): C 74.15, H 6.69; found C 74.06, H 6.68.

### St 35: (+)-6-O-Allyl-3,4,5-tri-O-benzyl-1-O-(4-methoxybenzyl)-2-O-(3,4,6-tri-O-benzyl-α-D-mannopyranosyl)-(1→2) -D-myo-inositol

Compound 34 (14 g, 12.5 mmol) was dissolved in 200 ml of methylamine solution (33% in anhydrous EtOH) and stirred for 6 h at room temperature. The reaction mixture was concentrated, diluted with toluene and evaporated. Silica gel column chromatography of the residue (petrol ether/EtOAc 4:1→3:1) afforded 35 (13 g) in quantitative yield. R_{f} (petrol ether/EtOAc 2:1)=0.45 [α]_{D}=+31 (c=1, CHCl₃). —¹H-NMR (250 MHz, CDCl₃) δ 2.41 (s, 1H, OH), 3.22-3.50 (m, 5H), 3.8 (s, 3H, OMe), 3.65-3.99, 4.02-4.2, 4.25-4.95 (m, 23H, CH₂CH═CH₂, CH₂-Ph, H_{Inositol}, H_{Man}), 5.14-5.35 (m, 2H, CH₂CH═CH₂), 5.39 (s, 1H, 1-H_{Man}), 5.9-6.09 (m, 1H, CH₂CH═CH₂), 6.83-6.91 (m, 2H, H_{PMB}), 7.1-7.45 (m, 32H, ArH). - Anal. calcd. for C₆₅H₇₀O₁₂*¼ H₂O (1047.76) C 74.51, H 6.78; found C 74.39, H 7.07.

### St 36: (+)-6-O-Allyl-3,4,5-tri-O-benzyl-1-O-(4-methoxybenzyl)-2-O-(2,3,4,6-tetra-O-benzyl-α-D-mannopyranosyl)-(1→2) -D-myo-inositol

To a solution of 35 (14 g, 13.42 mmol), in dry DMF (150 ml), were added BnBr (2.87 ml, 24.16 mmol) and NaH (580 mg, 24.16 mmol). After 3 h stirring at room temperature, the reaction mixture was concentrated in vacuo. The residue was dissolved in ethyl acetate, washed with water, brine and dried (MgSO₄). The organic layer was evaporated and the crude was purified by silica gel column chromatography (petrol ether/ethyl acetate 5:1) affording 36 (13.1 g, 86%). R_{f} (petrol ether/ethyl acetate 2:1)=0.74. −[α]_{D}=+12.8 (c=1, CHCl₃). —¹H-NMR (250 MHz, CDCl₃) δ 3.22-3.41 (m, 4H), 3.48-3.65 (m, 2H), 3.71 (s, 3H, OMe), 3.65-3.87 (m, 2H), 3.99-4.17 (m, 2H), 4.2-4.69, 4.7-4.92 (m, 20H, CH₂CH═CH₂, CH₂-Ph, H_{Inositol}, H_{Man}.), 5.12-5.36 (m, 2H, CH₂CH═CH₂), 5.42 (s, 1H, 1-H_{Man}), 5.91-6.10 (m, 1H, CH₂CH═CH₂), 6.76-6.86 (m, 2H, H_{PMB}), 7.07-7.40 (m, 37H, ArH). - Anal. calcd. for C₇₂H₇₆O₁₂ (1133.39): C 76.3, H 6.76; found C 76.09, H 6.77.

### St 37: (+)-3,4,5-Tri-O-benzyl-1-O-(4-methoxybenzyl)-6-O-[(2R/2S)-2,3-dihydroxypropan-1-yl]-2-O-(2,3,4,6-tetra-O-benzyl-α-D-mannopyranosyl)-D-myo-inositol

To a solution of 36 (14 g, 12.35 mmol), in a mixture of acetone-water (8:1), were added osmium tetroxide (100 mg, 0.39 mmol) and N-methylmorpholine-N-oxide (3.3 g, 24.42 mmol). After 14 h, saturated sodium bisulphite solution (170 ml) was added and the mixture was stirred for 30 min. The solution becomes dark and the solid was filtered. To the resulted clear solution was added ethyl acetate (2×200 ml). The combined organic extracts were dried (MgSO₄) and concentrated. Column chromatography of the residue (petrol ether/ethyl acetate 1:1→1:2) afforded 37a,b (14 g, 97%) as a colourless syrup. R_{f} (petrol ether/ethyl acetate 2:1)=0.11. −[α]_{D}=+11.8 (c=1, CDCl₃). —¹H-NMR (250 MHz, CDCl₃) δ 1.53 (s, 2H, OH), 1.89-1.99 (m, 2H, OH), 3.18-3.89 (m, 34H, OMe, OMe), 3.99-4.16 (m, 4H), 4.3-4.63, 4.68-4.80, 4.81-4.95 (m, 34H, CH₂-Ph), 5.34 (d, 1H, J<1 Hz, 1-H_{Man}), 5.36 (d, 1H, J=1.49 Hz, 1-H_{Man}), 6.72-6.83 (m, 4H, H_{PMB}), 7.09-7.39 (m, 74H, ArH). - Anal. calcd. for C₇₂H₇₈O₁₆ (1167.40) C 74.08, H 6.74; found C 74.01, H 6.88.

### St 38-1 : (+)-3,4,5-Tri-O-benzyl-1-O-(4-methoxybenzyl)-6-O-[(2S)-2-hydroxy-3-O-(tert-butyldiphenylsilyl)-propan-1-yl]-2-O-(2,3,4,6-tetra-O-benzyl-α-D-mannopyranosyl)-D-myo-inositol

To a solution of the both diastereomers 37a,b (12.87 g, 11.02 mmol), in dry dichloromethane (300 ml), imidazole (2.25, 33.05 mmol) and tertbutyldiphenylsilyl chloride (7.1 ml, 27.74 mmol) were added. The mixture was stirred for 3 h at 0° C., then washed with NH₄Cl-solution, water, dried (MgSO₄) and concentrated in vacuo. The residue was purified by flash chromatography (petrol ether/ethyl acetate 4:1) to gave 38-1 and 38-2 in quantitative yield. Separation of the two diastereomers were achieved by repeated flash chromatography (petrol ether/ethyl acetate 6:1). TLC: HPTLC-plates: 38-1 R_{f} (toluene/ethyl acetate 10:1) 0.45, 38-2 R_{f} (toluene/ethyl acetate 10:1)=0.40. −[α]_{D}+10.8 (c=1, CHCl₃). —¹H-NMR (600 MHz, CDCl₃) δ 1.05 (s, 9H, tBu), 3.21 (dd, 1H, J_{1,2}=1.2 Hz, J_{1,6}=9.82 Hz, 1-H_{Inositol}), 3.25 (dd, 1H, J_{3,4}=9.85 Hz, J_{3,2}=2.03 Hz, 3-H_{Inositol}), 3.32 (dd, 1H, J_{5,6}=J_{5,4}=9.3 Hz, 5-H_{Inositol}), 3.35-3.39 (m, 1H, 6-H_{Man}), 3.54 (dd, 1H, J_{vic.}=3.70 Hz, J_{gem.}=12.09 Hz, 6-H_{Man}), 3.75-3.68 (m, 6H, OMe, 3″, 6-H_{Inositol}), 3.69-3.75 (m, 2H, ²-H_{Man}, 4-H_{Inositol}), 3.78-3.82 (m, 2H, 3-H_{Man}, 2″), 3.87-3.94 (m, 2H, 1″), 4.04 (dd, 1H, J_{4,5}=J_{4,3}=9.7 Hz, 4-H_{Man}), 4.07-4.13 (m, 1H, 5-H_{Man}), 4.33-4.61, 4.70-4.80, 4.81-4.85 (m, 17H, CH₂-Ph, 2-H_{Inositol}), 5.35 (d, 1H, 1-H_{Man}), 6.65-6.67 (m, 2H, H_{PMB}), 7.03-7.43, 7.59-7.69 (m, 47H, ArH). - Anal. calcd. for C₈₈H₉₆O₁₄Si (1405.81): C 75.20, H 6.9; found C 75.32, H 6.89. 2-hydroxy-

### St 38-2: (+)-3,4,5-Tri-O-benzyl-1-O-(4-methoxybenzyl) -6-O-[(2R)-3-O-(tert-butyldiphenylsilyl)-propan-1-yl]-2-O-(2,3,4,6-tetra-O-benzyl-α-D-mannopyranosyl)-D-myo-inositol

[α]_{D}=+8.1 (c=1, CHCl₃). —¹H-NMR (600 MHz, CDCl₃) δ 1.05 (s, 9H, tBu), 3.2-3.29 (m, 2H, 1-H_{Inositol}, 3-H_{Inositol}), 3.3-3.45 (m, 2H, 5-H_{Inositol}, 6-H_{Man}), 3.51-3.77 (m, 9H, OMe, 3″, 6-H_{Man}, 6-H_{Inositol}, 4-H_{Inositol} 2-H_{Man}), 3.79-3.86 (m, 2H, 1″, 3-H_{Man}), 3.9 (m, 1H, 2″), 3.96 (dd, 1H, J_{vic.}=2.89 Hz, J_{gem.}=10.76 Hz, 1″), 4.0-4.12 (m, 2H, 4-H_{Man}, 5-H_{Man}) 4.3-4.5, 4.51-4.61, 4.69-4.78, 4.8-4.91 (m, 17H, CH₂-Ph, 2-H_{Inositol})r 5.35 (d, 1H, 1-H_{Man}), 6.72-6.74 (m, 2H, H_{PMB}), 7.06-7.43, 7.60-7.70 (m, 47H, ArH). - Anal. calcd. for C₈₈H₉₆O₁₄Si (1405.81) C 75.20, H 6.90; found C 74.95, H 6.90.

### St 40-1: (+)-3,4,5-Tri-O-benzyl-1-O-(4-methoxybenzyl)-6-O-[(2S)-3-O-(tert-butyldiphenylsilyl-2-O-methansulfonyl)-propan-1-yl]-2-O-(2,3,4,6-tetra-O-benzyl-α-D-mannopyranosyl)-D-myo-inositol

A solution of 38-1 (5.25 g, 3.74 mmol), in pyridine:dichloromethane (1:1, 80 ml), was treated at room temperature with methanesulphonyl chloride (0.87 ml, 11.20 mmol). Stirring was maintained for 3.5 h, diluted with CH₂Cl₂, washed with a saturated. aqueous solution of NaHCO₃ and water. The organic layer was dried (MgSO₄) and evaporated under vacuo. Silica gel column chromatography (petrol ether/ethyl acetate 3:1) afforded 40-1 (5.06 g, 93%) as a foam. TLC: petrol ether/ethyl acetate (3:1), R_{f}=0.33. −[α]_{D}=+15.5 (c=1, CHCl₃). —¹H-NMR (600 MHz, CDCl₃) δ 1.05 (s, 9H, tBu), 2.90 (s, 3H, OMs), 3.13-3.26 (dd, 1H, J_{1,2}=1.47 Hz, J_{1,6}=8.92 Hz, 1-H_{Inositol}), 3.19-3.26 (m, 2H, 5-H_{Inositol}, 3-H_{Inositol}), 3.37 (m, 1H, 6-H_{Man}), 3.46 (dd, 1H, J_{6,5}=J_{6,1}=9.43 Hz, 6-H_{Inositol}), 3. 55 (m, 1H, 6-H_{Man}), 3.63-3.74 (m, 5H, OMe, ²-H_{Man}, 4-H_{Inositol}), 3.77-3.85 (m, 2H, 3″, 3-H_{Man}), 3.88 (m, 1H, 1″), 3.98-4.12 (m, 3H, 1″, 4-H_{Man}, 5-H_{Man}), 4.3-4.9 (m, 18H, 2″, CH₂-Ph, 2-H_{Inositol}.), 5.34 (d, 1H, 1-H_{Man}), 6.68-6.75 (m, 2H, H_{PMB}), 7.05-7.45, 7.60-7.69 (m, 47H, ArH). - Anal. calcd. for C₈₉H₉₈O₁₄SSi (1483.9): C 72.00, H 6.66; found C 72.04, H 6.50.

### St 40-2: (+)-3,4,5-Tri-O-benzyl-1-O-(4-methoxybenzyl)-6-O-[(2R)-3-O-(tert-butyldiphenylsilyl-2-O-methansulfonyl)-propan-1-yl]-2-O-(2,3,4,6-tetra-O-benzyl-α-D-mannopyranosyl)-D-myo-inositol

In the same manner as described for 40-1 compound 38-2 (3.99 g, 2.84 mmol) gave 40-2 (4.15 g, 98%) as a colourless foam. [α]_{D}=+11.5 (c=1, CHCl₃). —¹H-NMR (600 MHz, CDCl₃) δ 1.04 (s, 9H, tBu), 2.87 (s, 3H, OMs), 3.13 (dd, 1H, J_{1,2}=1 47 Hz, J_{1,6}=8.92 Hz, 1-H_{Inositol}), 3.19-3.26 (m, 2H, 5-H_{Inositol}, 3-H_{Inositol}), 3.37 (m, 1H, 6-H_{Man}), 3.46 (dd, 1H, J_{6,5}=J_{6,1}=9.43 Hz, 6-H_{Inositol}), 3.55 (m, 1H, 6-H_{Man}), 3.63-3.74 (m, 5H, OMe, 2-H_{Man}, 4-H_{Inositol}), 3.77-3. 85 (m, 4H, 3″, 1″, 3-H_{Man}), 4.02-4.13 (m, 3H, 1″, 4-H_{Man}, 5-H_{Man}), 4.3-4.9, 4.52-4.66, 4.7-4.83, 4.83-4.90 (m, 18H, 2″, CH₂-Ph, 2-H_{Inositol}), 5.34 (d, 1H, 1-H_{Man}), 6.68-6.75 (m, 2H, H_{PMB}), 7.05-7.45, 7.60-7.69 (m, 47H, ArH). —¹³C-NMR (150.9 MHz, CDCl₃) δ 19.17 (1C, C(CH₃)₃), 26.78 (3C, C(CH₃)₃), 38.40 (1C, SO₂—CH₃), 55.14 (1C, OMe), 63.79 (1C, 3′-C), 68.95 (1C, 6-C_{Man}), 71.18 (1C, 2-C_{Inositol}), 71.85-75.83 (12C, CH₂Ph, 1′-C, 4-C_{Man}, 5-C_{Man}, 2-C_{Man}), 78.46 (1C, 3-C_{Inositol}), 79.04 (1C, 3-C_{Man}), 80.11 (1C, 1-C_{Inositol}), 80.95 (1C, 4-C_{Inositol}), 82.05 (1C, 6-C_{Inositol}), 82.12 (1C, 2′-C), 82.88 (1C, 5-C_{Inositol}), 98.38 (1C, 1-C_{Man}), 113.92 (2C, C_{PMB}), 127.3-138.37 (57C, Ph), 159.38 (1C, C_{OMe}). —Anal. calcd. for C₆₉H₉₈O₁₄SSi (1483.9): C 72.00, H 6.66; found C 71.81, H 6.70.

### St 41-1: (+)-3,4,5-Tri-O-benzyl-1-O-(4-methoxybenzyl)-6-O-[(2R)-2-azido-3-O-(tert-butyldiphenylsilyl)-propan-1-yl]-2-O-(2,3,4,6-tetra-O-benzyl-α-D-mannopyranosyl)-D-myo-inositol

To a solution of 41-1 (5.06 g, 3.41 mmol), in dry DMF (150 ml), was added sodium azide (3.33, 3.41 mmol) and the mixture was heated at 90° C. for 24 h, diluted with ether and washed with water. The aqueous layer was extracted with ether (3×200 ml), dried (MgSO₄) and concentrated. The residue was purified by flash chromatography (petrol ether/ethyl acetate 5:1) to gave 41-1 (4.1 g, 84%). TLC: petrol ether/ethyl acetate (5:1), R_{f}=0.24. −[α]_{D}=+18 (c=1, CHCl₃). —¹H-NMR (250 MHz, CDCl₃) δ 1.02-1.12 (s, 9H, tBu), 3.14-3.58 (m, 6H), 3.6-3.85 (m, 8H), 3.69 (s, 3H, OMe), 4.0-4.14 (m, 2H), 4.3-4.63, 4.69-4.92 (m, 17H, CH₂-Ph, 2-H_{Inositol}), 5.34 (d, 1H, 1-H_{Man}), 6.69-6.74 (m, 2H, H_{PMB}), 7.07-7.46, 7.62-7.72 (m, 47H, ArH). - Anal. calcd. for C₈₈H₉₅O₁₃N₃Si (1430.82): C 73.87, H 6.69, N 2.9; found C 73.63, H 6.78, N 2.48.

### St 41-2 : (+)-3,4,5-Tri-O-benzyl-1-O-(4-methoxybenzyl)-6-O-[(2S) -2-azido-3-O- (tert-butyldiphenylsilyl) -propan-1-yl]-2-O-(2,3,4,6-tetra-O-benzyl-α-D-mannopyranosyl)-D-myo-inositol

In the same manner as described for 41-1 compound 40-2 (4.12 g, 2.78 mmol) gave 41-2 (3.6 g, 93%) as a colourless syrup. −[α]_{D}=+5.3 (c=0.54, CHCl₃). —¹H-NMR (250 MHz, CDCl₃) δ 1.02-1.12 (s, 9H, tBu), 3.14-3.58 (m, 6H), 3.6-3.85 (m, 8H), 3.69 (s, 3H, OMe), 4.0-4.14 (m, 2H), 4.3-4.63, 4.69-4.92 (m, 17H, CH₂-Ph, 2-H_{Inositol}), 5.34 (d, 1H, 1-H_{Man}), 6.68-6.73 (m, 2H, H_{PMB}), 7.07-7.46, 7.62-7.72 (m, 47H, ArH). - Anal. calcd. for C₈₈H₉₅O₁₃N₃Si (1430.82): C 73.87, H 6.69, N 2.9; found C 73.96, H 6.97, N 2.6. - MALDI: calcd. M+Sodium m/z=1453.82; found m/z=1453.3.

### St 43-1 : (+)-3,4,5-Tri-O-benzyl-1-O-(4-methoxybenzyl)-6-O-[(2S)-2-azido-3-hydroxy-propan-1-yl]-2-O-(2,3,4,6-tetra-O-benzyl-α-D-mannopyranosyl)-D-myo-inositol

To a solution of 41-1 (3.7 g, 2.59 mmol), in THF (100 ml), was added at 0° C. a tetrabutylammonia fluoride solution (1M in THF, 0.9 ml). The reaction mixture was allowed to reach room temperature. After 3.5 h, the reaction mixture was diluted with ethyl acetate, washed with NH₄Cl— solution, H₂O, dried (MgSO₄) and concentrated in vacuo. Silica gel column chromatography of the residue (petrol ether/ethyl acetate 3:1) afforded 43-1 (2.93 g, 95%) as a colourless oil. TLC: petrol ether/ethyl acetate (3:1), R_{f}=0.14. −[α]_{D}+13.8 (c=1, CHCl₃). —¹H-NMR (250 MHz, CDCl₃) δ 1.99 (dd, 1H, J=7.14 Hz, OH), 3.20-3.90 (m, 17H, OMe, 1-H_{Inositol}, 3-H_{Inositol}, 5-H_{Inositol}, 6-H_{Man}, 6-H_{Inositol}, 2″, 3″, 4-H_{Inositol}, 2-H_{Man}), 4.0-4.18 (m, 2H, 4-H_{Man}, 5-H_{Man}), 4.32-4.66, 4.7-4.95 (m, 17H, CH₂-Ph, 2-H_{Inositol}), 5.38 (d, 1H, J=1.26 Hz, 1-H_{Man}), 6.76-6.84 (m, 2H, H_{PMB}), 7.10-7.40 (m, 37H, ArH). - Anal. calcd. for C₇₂H₇₇O₁₃N₃ (1192.42): C 72.52, H 6.51, N 3.52; found C 72.38, H 6.30, N 2.99. - MALDI: calcd. M+Sodium m/z=1215.42; found m/z=1214.0.

### St 43-2: (+)-3,4,5-Tri-O-benzyl-1-O-(4-methoxybenzyl)-6-0-[(2R)-2-azido-3-hydroxy-propan-1-yl]-2-O-(2,3,4,6-tetra-O-benzyl-α-D-mannopyranosyl)-D-myo-inositol

In the same manner as described for 43-1 compound 42-2 (3.59 g, 2.51 mmol) gave 43-2 (2.8 g, 93%). −[α]_{D}=+19.2 (c=1, CHCl₃). —¹H-NMR (600 MHz, CDCl₃) δ 1.9 (s, 1H, OH), 3.23 (dd, 1H, J_{1,2}=1.44 Hz, J_{1,6}=9.71 Hz, 1-H_{Inositol}), 3.26 (dd, 1H, J_{3,4}=9.87 Hz, J_{3,2}=2. 17 Hz, 3-H_{Inositol}), 3.33 (dd, 1H, J_{5,6}=J_{5,4}=9.21 Hz, 5-H_{Inositol}), 3.36-3.41 (m, 1H, 6-H_{Man}), 3.45 (dd, 1H, J_{6,1}=J_{6,5}=9.5 Hz, 6-H_{Inositol}). 3.52-3.58 (m, 2H, 2″, 6-H_{Man}), 3.59-3.77 (m, 7H, OMe, 2-H_{Man}, 3″, 4-H_{Inositol}), 3.80-3.88 (m, 3H, 1″, 3-H_{Man}), 4.05 (dd, 1H, J_{4,5}=J_{4,3}=9.81 Hz, 4-H_{Man}), 4.13 (m, 1H, 5-H_{Man}), 4.34-4.41, 4.41-4 .46, 4.71-4.78, 4.84-4. 94 (m, 17H, CH₂-Ph, 2-H_{Inositol}), 5.37 (d, 1H, 1-H_{Man}), 6.78-6.83 (m, 2H, H_{PMB}), 7.10-7.38 (m, 37H, ArH). —¹³C-NMR (150.9 MHz, CDCl₃) δ 55.21 (1C, OMe), 62.58 (1C, 2′-C), 62.77 (1C, 3′-C), 68.94 (1C, 6-C_{Man}), 71.11 (1C, 2-C_{Inositol}), 71.82-75.93 (12C, CH₂Ph, 1′-C, 4-C_{Man,} 5-C_{Man,} 2-C_{Man}), 78.63 (1C, 3-C_{Inositol}), 79.16 (1C, 3-C_{Man}), 80.27 (1C, 1-C_{Inositol}), 81.08 (1C, 4-C_{Inositol}), 81. 96 (1C, 6-C_{Inositol}), 83.08 (1C, 5-C_{Inositol}), 98.35 (1C, 1C_{Man}), 113.96 (2C, C_{PMB}), 127.3-138.79 (45C, Ph), 159.52 (1C, C_{OMe}). - Anal. calcd. for C₇₂H₇₇O₁₃N₃ (1192. 42): C 72. 50, H 6. 50, N 3.52; found C 72.42, H 6.26, N 3.28.

### St 47-1: (+)-3,4,5-Tri-O-benzyl-1-O-(4-methoxybenzyl)-6-O-[(2R)-2-azido-propionicacidbenzylester]-2-O-(2,3,4,6-tetra-O-benzyl-α-D-mannopyranosyl)-D-myo-inositol

A solution of 43-1 (2.9 g, 2.43 mmol), in acetone (20 ml), at 0° C. was added to an aqueous 5% NaHCO₃-solution (10 ml). To this heterogeneous mixture was treated with KBr (295 mg, 2.45 mmol) and TEMPO (427 mg, 1.67 mmol). Sodium hypochlorite (NaOCl 13%, 5 ml) was added dropwise over 5 min and the mixture was stirred at 0° C. for further 10 min. The reaction mixture was diluted with H₂O and extracted with ethyl acetate (3×50 ml). The combined organic layers were washed with brine, dried (MgSO₄), concentrated in vacuo and placed under high vacuum for 1 h. R_{f} (toluene/acetone 1:1)=0.26. This material was directly used in the next step without further purification. To a solution of this free carboxylic acid in dry DMF (50 ml) were added benzyl bromide (0.59 ml, 4.97 mmol) and CsF (754 mg, 4.97 mmol) at room temperature and stirred for 3 h. The reaction mixture was diluted with ethyl acetate and washed with NH₄Cl-solution, brine, dried (MgSO₄) and concentrated. The residue was purified by silica gel column chromatography (petrol ether/EtOAc 4:1) to afford 47-1 (2.55 g, 81%) as a colourless syrup. TLC: (petrol ether/EtOAc 3:1), R_{f}=0.41. −[α]_{D}=+18.8 (c=1, CHCl₃). —¹H-NMR (600 MHz, CDCl₃) δ 3.18 (dd, 1H, J_{1,2}=1.33 Hz, J_{1,6}=9.63 Hz, 1-H_{Inositol}), 3.23 (dd, 1H, J_{3,4}=9.85 Hz, J_{3,2}=2.05 Hz, 3-H_{Inositol}), 3.32 (dd, 1H, J_{5,6}=J_{5,4}=9.22 Hz, 5-H_{Inositol}), 3.34-3.38 (m, 1H, 6-H_{Man}), 3.47 (dd, 1H, J_{6,1}=J_{6,5}=9.41 Hz, 6-H_{Inositol}), 3.54 (dd, 1H, J_{gem.}=10.71 Hz, J_{vic.}=3.5 Hz, 6-H_{Man}), 3.64 (m, 1H, 2-H_{Man}), 3.69 (s, 3H, OMe), 3.72 (dd, 1H, J_{4,5}=J_{4,3}=9.53 Hz, 4-H_{Inositol}), 3.81 (dd, 1H, J_{3,4}=9.43 Hz, J_{3,2}=2.97 Hz, 3-H_{Man}), 4.01 (dd, 1H, J_{vic.}=5.44 Hz, 2″), 4.05 (dd, 1H, J_{4,5}=J_{4,3}=9.61 Hz, 4-H_{Man}), 4.07-4.13 (m, 3H, 1″, 5-H_{Man}), 4.29-4.39, 4.41-4.48, 4.51-4.62, 4.7-4.79, 4.82-4.92 (m, 17H, CH₂-Ph, 2-H_{Inositol}), 5.12 (d, 1H, J_{gem.}=12.21 Hz, COOCH₂-Ph), 5.23 (d, 1H, J_{gem.}=12.21 Hz, COOCH₂-Ph), 5.34 (d, 1H, 1-H_{Man}), 6.72-6.8 (m, 2H, H_{PMB}), 7.10-7.40 (m, 42H, ArH). - Anal. calcd. for C₇₉H₈₁O₁₄N₃ (1296.52): C 73.19, H 6.30, N 3.24; found C 73.03, H 6.28, N 2.73. - MALDI: calcd. M+Sodium m/z=1319.52; found m/z=1318.3.

### St 47-2: (+)-3,4,5-Tri-O-benzyl-1-O-(4-methoxybenzyl)-6-O-[(2S)-2-azido-propionicacidbenzylester]-2-O-(2,3,4,6-tetra-O-benzyl-α-D-mannopyranosyl)-D-myo-inositol

In the same manner as described for 47-1 compound 43-2 (2.75 g, 2.31 mmol) gave 47-2 (2.33 g, 78%). −[α]_{D}=+20.5 (c=1, CHCl₃). —¹H-NMR (250 MHz, CDCl₃) δ 3.20-3.39 (m, 4H, 1-H_{Inositol}, 3-H_{Inositol}, 6-H_{Man}), 3.42-3.58 (m, 2H, 6-H_{Inositol}, 6-H_{Man}), 3.61-3.67 (m, 1H, 2-H_{Man}), 3.69 (s, 3H, OMe), 3.70-3.84 (m, 2H, 4-H_{Inositol}, 3-H_{Man}), 3.93-4.23 (m, 5H, 2″, 4-H_{Man}, 1″, 5-H_{Man}), 4.30-4.63, 4.69-4.92 (m, 17H, CH₂-Ph, 2-H_{Inositol}), 5.02 (d, 1H, J_{gem.}=12.20 Hz, COOCH₂-Ph), 5.23 (d, 1H, J_{gem.}=12.21 Hz, COOCH₂-Ph), 5.34 (d, 1H, J<1 Hz, 1-H_{Man}), 6.73-6.81 (m, 2H, H_{PMB}), 7.09-7.40 (m, 42H, ArH). - Anal. calcd. for C79H₈₁O₁₄N₃ (1296.52): C 73.19, H 6.30, N 3.24; found C 73.12, H 6.42, N 2.80.

### St 48-1: (+)-3,4,5-Tri-O-benzyl-6-O-[(2R)-2-azido-propionicacidbenzylester]-2-O-(2,3,4,6-tetra-O-benzyl-α-D-mannopyranosyl)-D-myo-inositol

A solution of 47-1 (1 g, 0.77 mmol), in acetonitrile:toluene:water (60:3:4, 40 ml), was cooled to 0° C. and treated with Ce(NH₄)₂(NO₃)₆(2.1 g, 3.83 mmol). After ½ hour at 0° C., the reaction was allowed to reach room temperature. The mixture was stirred for 1.5 h, diluted with EtOAc, washed with saturated aqueous NaHCO₃-solution, dried (MgSO₄) and concentrated. Flash chromatography (petrol ether/EtOAc 4:1) of the residue gave 48-1 (0.77 g, 85%) as a colourless syrup. TLC: (petrol ether/EtOAc 2:1), R_{f}=0.51. −[α]_{D}+43 (c=1, CHCl₃). —¹H-NMR (250 MHz, CDCl₃) δ 2.88 (d, 1H, J<1 Hz, OH), 3.18-3.40 (m, 5H), 3.55 (dd, 1H, J_{gem.}=11. 43 Hz, 6-H_{Man}), 3.62-3.85 (m, 4H), 3.93-4.01 (m, 1H), 4.02-4.20 (m, 3H), 4.27-4.96 (m, 15H), 5.28 (s, 2H, COOCH₂-Ph), 5.43 (d, 1H, J<1 Hz, 1-H_{Man}), 7.09-7.41 (m, 40H, ArH). - Anal. calcd. for C₇₁H₇₃O₁₃N₃ (1176.37): C 72.49, H 6.26, N 3.57; found C 72.32, H 6.38, N 3.00. - MALDI: calcd. M+Sodium m/z=1199.37; found m/z=1198.9.

### St 48-2: (+)-3,4,5-Tri-O-benzyl-6-O-[(2S)-2-azido-propionicacidbenzylester]-2-O-(2,3,4, 6-tetra-O-benzyl-α-D-mannopyranosyl)-D-myo-inositol

In the same manner as described for 48-1 compound 47-2 (1.03 g, 0.79 mmol) gave 48-2 (757 mg, 81%) as a colourless syrup. −[α]_{D}+15.8 (c=1, CHCl₃). —¹H-NMR (250 MHz, CDCl₃) δ 3.10 (d, 1H, J=1.52 Hz, OH), 3.18-3.41 (m, 5H), 3.58 (dd, 1H, J_{gem.}=11.96 Hz, 6-H_{Man}), 3.65-3.77 (m, 3H), 3.78-3.89 (m, 1H), 4.02-4.13 (m, 3H), 4.19-4.28 (m, 1H), 4.29-4.95 (m, 15H), 5.20 (d, 1H, J=9.24 Hz, COOCH₂-Ph), 5.30 (d, 1H, J=10.35 Hz, COOCH₂-Ph), 5.46 (d, 1H, J=1.6 Hz, 1-H_{Man}), 7.12-7.43 (m, 40H, ArH). - Anal. calcd. for C₇₁H₇₃O₁₃₃N₃ (1176.37): C 72.49, H 6.26, N 3.57; found C 72.33, H 6.36, N 2.95.

### St 49-1,a,b : (+)-3,4,5-Tri-O-benzyl-6-O-[ (2R)-2-azido-propionicacidbenzylester]-2-O- (2,3,4,6-tetra-O-benzyl-α-D-mannopyranosyl)-D-myo-inosit-1-yl-[benzyloxyl-[(2R) -2,3-bis-(myristoyloxy)-propyl]-phosphate

Terazole (93 mg, 1.33 mmol) was dried for 1 h under high vacuum. Compound 48-1 (600 mg, 0.51 mmol) was dissolved in anhydrous dichloromethane (20 ml), added to the tetrazole and stirred at rt under argon atmosphere. To this reaction mixture was added dropwise benzyl N,N-diisopropylphosphoramidite (765 mg, 1.02 mmol) and stirred for 2 h. R_{f} (petrol ether/EtOAc 4:1)=0.6. Then treated with tertbutylperoxide (4.7 M in isooctane, 1 ml). After 15 min, the reaction mixture was diluted with CH₂Cl₂, 5% sodium bisulphite-solution was added and the two layers were separated. The organic layer was washed with brine, dried (MgSO₄) and concentrated. The residue was purified by flash chromatography (petrol ether/EtOAc 4:1) (toluene/EtOAc 10:1) to afford 49-1 (732 mg, 78%) as a colourless syrup. TLC: (petrol ether/EtOAc 4:1), R_{f}=0.27. −[α]_{D}=+12.7 (c=1, CHCl₃). —³¹P-NMR (600 MHz, CDCl₃) δ 0.1 (s, 1P), 0.402 (s, 1P). —¹H-NMR (600 MHz, CDCl₃) δ 0.8-0.92 (t, 12H, Me), 1.08-1.40 (s, 80H, CH₂-Chain), 1.44-1.67 (m, 8H, COCH₂CH₂R), 2.12-2.36 (m, 8H, COCH₂CH₂R), 3.21-3.38 (m, 6H, 3-H_{Inositol}, 5-H_{Inositol}), 3.45-3.56 (m, 6H, 6-H_{Inositol}, 6-H_{Man}), 3.68-3.77 (m, 4H, 4-H_{Inositol}, 2-H_{Man}), 3.82 (dd, 2H, J_{3,4}=9.36 Hz, J_{3,2}=2.5 Hz, 3-H_{Man}), 3.88-4.35 (m, 16H, 1″, 1′, 2′, 4-H_{Man}, 1-H_{Inositol}, 5-H_{Man}), 4.40-4.92, (m, 30H, 2-H_{Inositol}, CH₂-Ph), 5.03-5.28 (m, 14H, 2″, 3″, COOCH₂-Ph, POCH₂Ph), 5.32 (d, 1H, J<1 Hz, 1-H_{Man}), 5.34 (d, 1H, J<1 Hz, 1-H_{Man}), 7.03-7.42 (m, 90H, ArH). - MALDI: calcd. M+Sodium m/z=1864.4; found m/z=1865.4.

### St 49-2,a,b: (+)-3,4,5-Tri-O-benzyl-6-O-[(2S)-2-azido-propionicacidbenzylester]-2-O-(2,3,4,6-tetra-O-benzyl-α-D-mannopyranosyl)-D-myo-inosit-1-yl-[benzyloxy]-[(2R)-2,3-bis-(myristoyloxy)-propyl]-phosphate

In the same manner as described for 49-1 compound 48-2 (417 mg, 0.35 mmol) gave 48-2 (490 mg, 75%) as a colourless oil. −[α]_{D}=+7 (c=1, CHCl₃). —¹H-NMR (250 MHz, CDCl₃) δ 0.8-0.95 (t, 12H, Me), 1.14-1.39 (s, 80H, CH₂-Chain), 1.46-1.56 (m, 8H, COCH₂CH₂R), 2.15-2.35 (m, 8H, COCH₂CH₂R), 3.20-3.38 (m, 6H), 3.45-3.59 (m, 4H), 3.65-3.85 (m, 6H), 3.93-4.32 (m, 16H), 4.40-4.92 (m, 30H), 4.40-4.92, (m, 30H), 5.04-5.25 (m, 14H, 2″, 3″, COOCH₂-Ph, POCH₂Ph), 5.31 (d, 1H, J<1 Hz, 1-H_{Man}), 5.34 (d, 1H, J<1 Hz, 1-H_{Man}), 7.10-7.41 (m, 90H, ArH).

### St 50-1 : 6-O-[(2S)-2-amino-propionicacid]-2-O-α-D-mannopyranosyl-D-myo-inosit-1-yl-[(2R)-2,3-bis-(myristoyloxy)-propyl]-phosphate

A vigorously stirred mixture of 49-1 (250 mg, 0.14 mmol) CH₂Cl₂:MeOH:H₂O (7.5:7.5:1, 3 ml) and Pearlman's catalyst (0.2 equiv.) was degassed under vacuum and saturated with hydrogen (by a H₂-filled balloon) three times. The suspension was stirred at room temperature over night, filtered over celite and washed with CH₂Cl₂:MeOH:H₂O (7.5:7.5:1, 2 ml). The solvents were removed under vacuum to afford 50-1 (110 mg, 81%) as a white solid. —³¹P-NMR (600 MHz, dmso) δ 0.909 (s, 1P). —¹H-NMR (600 MHz, dmso) δ 0.78-0.90 (t, 6H, Me), 1.05-1.35 (s, 40H, CH₂-Chain), 1.40-1.56 (m, 4H, COCH₂CH₂R), 2.18-2.33 (m, 4H, COCH₂CH₂R), 3.14 (m, 1H, 5-H_{Inositol}), 3.19 (m, 1H, 3-H_{Inositol}), 3.34 (m, H,4-H_{Inositol}), 3.35 (m, 1H, 6-H_{Inositol}), 3.47 (m, 2H, 6-H_{Man}, 4-H_{Man}), 3.49 (m, 1H, 3-H_{Man}), 3.57 (m, 1H, 6-H_{Man}), 3.66 (m, 1H, ²-H_{Man}), 3.74 (m, 1H, 1′), 3.83 (m, 2H, 1″), 3.86 (m, 1H, 5-H_{Man}), 3.95 (m, 1H, 2-H_{Inositol}), 3.97 (m, 1H, 2′), 4.02 (m, 1H, 1-H_{Inositol}), 4.09 (m, 1H, 3″), 4.27 (m, 2H, 1′, 3″), 4.98 (d, 1H, J<1 Hz, 1-H_{Man}), 5.10 (m, 1H, 2″), 8.65-8.9 (bs, 2H, NH₂). - Anal. calcd. for C₄₆H₈₆O₂₀NP*2.5 H₂O (1049.16): C 52.66, H 8.74, N 1.33; found: C 52.79, H 8.94, N 1.03.

### St 50-2 : 6-O-[(2R)-2-amino-propionicacid]-2-O-α-D-mannopyranosyl-D-myo-inosit-1-yl-[(2R)-2,3-bis-(myristoyloxy)-propyl]-phosphate

In the same manner as described for 50-1 compound 49-2 (250 mg, 0.14 mmol) gave 50-2 (120 mg, 88%) as a white solid. —³¹P-NMR (600 MHz, dmso) δ 0.847 (s, 1P). —¹H-NMR (600 MHz, dmso) δ 0.76-0.90 (t, 6H, Me), 1.07-1.35 (s, 40H, CH₂-Chain), 1.42-1.55 (m, 4H, COCH₂CH₂R), 2.18-2.35 (m, 4H, COCH₂CH₂R), 3.11 (m, 1H, 5-H_{Inositol}), 3.19 (m, 1H, 3-H_{Inositol}), 3.34 (m, 2H, 6-H_{Inositol}, 4-H_{Inositol}), 3.48 (m, 2H, 6-H_{Man}, 4-H_{Man}), 3.49 (m, 1H, 3-H_{Man}), 3.56 (m, 1H, 6-H_{Man}), 3.66 (m, 1H, 2-H_{Man}), 3.86 (m, 3H, 1″, 5-H_{Man}), 3.92 (m, 1H, 1′), 3.93 (m, 1H, 2′), 3.94 (m, 1H, 2-H_{Inositol}), 3-98 (m, 1H, 1-H_{Inositol}), 4.09 (m, 1H, 3″), 4.19 (m, 1H, 1′), 4.28 (m, 1H, 3″), 4.97 (d, 1H, J<1 Hz, 1-H_{Man}), 5.11 (m, 1H, 2″), 8.67 (bs, 2H, NH₂). - Anal. calcd. for C₄₆H₈₆O₂₀NP*3.5 H₂O (1067.16): C 51.77, H 8.78, N 1.31; found: C 51.74, H 9.00, N 0.98.

### St 10: (+)-1-O-Benzyl-2,3:5,6-di-O-cyclohexyliden-D-myo-inositol

A mixture of 9 (950 mg, 2.21 mmol), anhydrous THF (25 ml), Pearlman's catalyst (10 mol %) was degassed under vacuum and saturated with hydrogen three times. The suspension was stirred at room temperature for 2 h, then quenched with triethylamine, filtered over celite, washed with THF and concentrated. Compound 10 (736 mg, 98%) was obtained as a white solid. R_{f} (petrol ether/ethyl acetate 1:1) 0.32. −M.p.: 190° C. −[α]_{D}=+17.3 (c=0.75, CHCl₃). —¹H-NMR (250 MHz, CDCl₃) δ 1.25-1.86 (m, 20H, H_{Cyclo}.) 3.39-2.7 (2s, 2H, OH), 3.32 (dd, 1H, J=10.66 Hz, J=9.33 Hz, 5-H_{Inositol}), 3.72-3.93 (m, 2H), 3.95-4.12 (m, 2H), 4.49 (dd, 1H, J_{2,3}=J_{2,1}=4.83 Hz, 2-H_{Inositol}). - Anal. calcd. for C₁₈H₂₈O₆*¼ H₂O (344.92): C 62.68, H 8.33; found: C 62.76, H 8.35.

### St 11: (+)-2,3:5,6-Di-O-cyclohexyliden-1,4-di-O-methyl-D-myo-inositol

To a solution of compound 10 (440 mg, 1.29 mmol), in dry DMF (5 ml), were added methyl iodide (200 μl, 3.2 mmol) and sodium hydride (116 mg, 4.8 mmol). After 3 h stirring at room temperature, the reaction mixture was diluted with saturated aqueous NH₄Cl-solution and dichloromethane. The organic layer was washed with water (twice), dried (MgSO₄) and concentrated. The residue was purified by column chromatography (petrol ether/EtOAc 2:1) to afford 11 (438 mg, 92%) as a white solid. R_{f} (petrol ether/ethyl acetate 1:1)=0.76. −M.p.: 118° C. −[α]_{D}=+8 (c=1, CHCl₃). —¹H-NMR (600 MHz, CDCl₃) δ 1.25-1.5, 1.51-1.82 (m, 20H, H_{Cyclo}.), 3.31 (dd, 1H, J_{5,6}=J_{5,4}=9.77 Hz, 5-H_{Inositol}), 3.47 (dd, 1H, J_{4,5}=10.55 Hz, J_{4,3}6.44 Hz, 4-H_{Inositol}), 3.58 (s, 3H, OMe), 3.60 (s, 3H, OMe),), 3.62 (dd, 1H, J_{1,2}=4.2 Hz, J_{1,6}=10.14 Hz, 1-H_{Inositol}), 3.96 (dd, 1H, J_{6,1}=J_{6,5}=9.76 Hz, 6-H_{Inositol}), 4.06 (dd, 1H, J_{3,4}=J_{3,2}=5.6 Hz, 3-H_{Inositol}), 4.52 (dd, 1H, J_{2,3}=J_{2,1}=4.55 Hz, 2-H_{Inositol}). - Anal. calcd. for C₂₀H₃₂O₆ (368.47): C 65.19, H 8.75; found: C 65.16, H 8.75.

### St 12: (-)-1,4-Di-O-methyl-D-myo-inositol (−)-Liriodentritol

To a solution of 11 (160 mg, 0.41 mmol), in MeOH:CH₂Cl₂ (1:1, 2 ml), was added camphor-10-sulfonic acid (15 mg, 0.065 mmol) at room temperature and stirred for 24 h. The white precipitate, which was formed, was filtered, washed with dichloromethane and dried under high vacuum. Compound 12 was obtained in 78% yield (67 mg). R_{f} (CHCl₃/MeOH 5:1)=0.07. −M.p.: 226° C. −[α]_{D}=−25 (c=1.5, H₂O) −[α]_{D}=−25 (c=2, H₂0). —¹H-NMR (600 MHz, dmso) δ 2.78 (dd, 1H, J_{1,2}=2.47 Hz, J_{1,6}=9.63 Hz, 1-H_{Inositol}),3.0 (ddd, 1H, J_{5,6}=J_{5,4}=9.14 Hz, J_{5-OH}=4.69 Hz, 5-H_{Inositol}), 3.08 (dd, 1H, J_{4,5}=J_{4,3}=9.38 Hz, 4-H_{Inositol}), 3.18 (ddd, 1H, J_{3,4}=9.38 Hz, J_{3,2}=2.72 Hz, J_{3-OH}=6.42 Hz, 3-H_{Inositol}), 3.28 (s, 3H, 1-OMe), 3.43 (s, 3H, 4-OMe), 3.43 (ddd, 1H, J_{6,1}=9.63 Hz, J_{6,5}=9.14 Hz, J_{6-OH}=4-94 Hz, 6-H_{Inositol}), 3.88 (ddd, 1H, J_{2,3}=2.72 Hz, J_{2,1}=2.47 Hz, J_{6-OH}=3.95 Hz, 2-H_{Inositol}), 4.54 (d, 1H, J=6.67 Hz, 3-OH), 4.56 (d, 1H, J=3.95 Hz, 2-OH), 4.58 (d, 1H, J=4.94 Hz, 6-OH), 4.65 (d, 1H, J=4.69 Hz, 5-OH). —¹³C-NMR (150.9 MHz, dmso d₆) δ 56.59 (1C, OMe_{1-c}), 56.68 (1C, OMe_{4-c}), 68.53 (1C, 2-C), 71.08 (1-C, 3-C), 71.93 (1C, 6-C), 74.67 (1C, 5-C), 81.39 (1C, 1-C), 83.10 (1C, 4-C). - Anal. calcd. for C₈H₁₆O₆ (208.21): C 46.14, H 7.75; found: C 46.00, H 7.71.

### St 25: (+)-3,4,5-Tri-O-benzyl-1-O-(4-methoxybenzyl)-2-O-(2,3,4, 6-tetra-O-benzyl-α-D-mannopyranosyl)-(1→2) -L-myo-inositol

Compound 24 (3 g, 2.88 mmol) was dissolved in EtOH 90° C. (45 ml) by heating, then DBU (43 μl, 0.29 mmol) and (Ph₃P)₃RhCl (750 mg, 0.81 mmol) were added. The mixture was stirred for 1.5 h under reflux and then concentrated in vacuo (propenyl, R_{f}=0.54, petrol ether/EtOAc 2:1). The residue was dissolved in 1:9 1M HCl/acetone (50 ml), and the solution was heated under reflux for 15 min. Acidity was neutralized by adding triethylamine, then the mixture was diluted with ethyl acetate, washed with water, dried (MgSO₄) and concentrated. Flash chromatography (petrol ether/EtOAc 4:1→5:2) of the residue gave 25 (2.5 g, 87%). R_{f} (petrol ether/EtOAc 2:1)=0.38. −[α]_{D}=+8.5 (c=0.25, CHCl₃). —¹H-NMR (600 MHz, CDCl₃) δ 2.39 (s, 2H, OH), 3.06 (dd, 1H, J_{1,2}=2.35 Hz, J_{1,6}=9.98 Hz, 1-H_{Inositol}), 3.32 (dd, 1H, J_{5,6}=J_{5,4}=9.1 Hz, 5-H_{Inositol}), 3.34 (dd, 1H, J_{3,4}=10.0 Hz, J_{3,2}=2.35 Hz, 3-H_{Inositol}), 3.42 (dd, 1H, J_{gem.}=10-35 Hz, J_{vic.}=2.05 Hz, 6-H_{Man}), 3.56 (dd, 1H, J_{gem.}=10.56 Hz, J_{vic.}=3.81 Hz, 6-H_{Man}), 3.77 (s, 3H, OMe), 3.8 (dd, 1H, J_{4,5}=J_{4,3}=9.39 Hz, 4-H_{Inositol}), 3.84 (dd, 1H, J_{3,4}=9.39 Hz, J_{3,2}=3.23 Hz, 3-H_{Man}), 3.9 (dd, 1H, J_{4,5}=J_{4,3}=9. 68 Hz, 4-H_{Man}), 3.94 (dd, 1H, J_{6,1}=J_{6,5}=9. 68 Hz, 6-H_{Inositol}), 4.05 (dd, 1H, J_{2,3}=<1 Hz, 2-H_{Man}), 4.15 (ddd, 1H, J_{vir.}=1.76 Hz, J_{vic.}=3.52 Hz, J_{5,4}=9.98 Hz, 5-H_{Man}), 4.35 (dd, 1H, J_{2,3}=J_{2,1}=2.35 Hz, 2-H_{Inositol}), 4.36-4.47, 4.56-4.61, 4.63-4.73, 4.77-4.88 (m, 14H, CH₂-Ph), 5.37 (d, 1H, J<1, 1-H_{Man}), 6.78-6.80 (m, 2H, H_{PMB}), 7.08-7.39 (m, 32H, ArH). —¹³C-NMR (150.9 MHz, CDCl₃) δ 55.24 (1C, OMe), 68.65 (1C, ²-C_{Man}), 68.79 (1C, 6-C_{Man}), 71.02 (1C, 5-C_{Man}), 71.35-75.79 (10C, CH₂Ph, 2-C_{Inositol,}6-C_{Inositol,}4-C_{Man}), 77.63 (1C, 1-C_{Inositol}), 79.68 (1C, 3-C_{Man}), 80.81 (1C, 3-C_{Inositol}), 81.28 (1C, 4-C_{Inositol}), 83.31 (1C, 5-C_{Inositol}) 100.0 (1C, 1-C_{Man}), 113.86 (2C, C_{PMB}), 127.52-138.62 (39C, Ph), 159.18 (1C, C_{OMe}). - Anal. calcd. for C₆₂H₆₆O₁₂*1/4 H₂O (1007.7): C 73.9, H 6.65; found C 73.81, H 6.67.

### St 56: (+)-3,4,5-Tri-O-benzyl-1-O-(4-methoxybenzyl)-6-O-[(2R)-2-hydroxy-3-O-(tert-butyldiphenylsilyl)-propan-1-yl]-2-O-(2,3,4,6-tetra-O-benzyl-α-D-mannopyranosyl)-L-myo-inositol

To a solution of 31 (40 mg, 0.037 mmol), in anhydrous DMF (2.5 ml), was added under argon atmosphere the sulphate (143.6 mg, 0.37 mmol) and sodium hydride (4 mg, 0.17 mmol). The reaction mixture was followed by TLC (R_{f} (EtOAc/MeOH 18:1)=0.30), R_{f} (petrol ether/ethyl acetate 2:1)=0. After 4 h stirring at rt a second portion of sulphate (70 mg, 0.18 mmol) and sodium hydride (10 mg, 0.42 mmol) were added. Stirring was maintained for 20 h before the reaction mixture was concentrated. The residue was purified by silica gel column chromatography (EtOAc/MeOH 18:1) to afford the sodium salt. This compound was dissolved in dioxane (2 ml), acidified with 0. 1 M H₂SO₄ and stirred at rt for 1 h, diluted with EtOAc and washed with saturated aqueous NaHCO₃-solution. The organic layer was dried (MgSO₄) and concentrated. Flash chromatography afforded 56 (26 mg, 51%) as a colourless syrup. R_{f} (petrol ether/ethyl acetate 2:1)=0.72. −[α]_{D}=+24.5 (c=1, CHCl₃). —¹H-NMR (600 MHz, CDCl₃) δ 1.00 (s, 9H, tBu), 3.14 (dd, 1H, J_{1,2}=2.17 Hz, J_{1,6}=7.83 Hz, 1-H_{Inositol}), 3.23-3.29 (m, 1H, 3-H_{Inositol}), 3.31 (dd, 1H, J_{5,6}=J_{5,4}=7.92 Hz, 5-H_{Inositol}), 3.44 (dd, 1H, J_{gem.}=10.21 Hz, 6-H_{Man}), 3.54-3.74 (m, 9H, OMe, 3″, 6-H_{Man}, 6-H_{Inositol}, 4-H_{Inositol} 2-H_{Man}), 3.76-3.84 (m, 2H, 2″, 3-H_{Man}), 3.86-3.93 (m, 2H, 1″), 4.0-4.09 (m, 2H, 4-H_{Man}, 5-H_{Man}), 4.32-4.65, 4.70-4.88 (m, 17H, CH₂-Ph, 2-H_{Inositol}), 5.40 (d, 1H, 1-H_{Man}), 6.66-6.73 (m, 2H, H_{PMB}), 7.03-7.45, 7.56-7.68 (m, 47H, ArH). - Anal. calcd. for C₈₈H₉₆O₁₄Si*½ H₂O (1414.82): C 74.71, H 6.91; found C 74.73, H 6.82.

### St 57: (+)-3,4,5-Tri-O-benzyl-1-O-(4-methoxybenzyl)-6-O-[(2R)-3-O-(tert-butyldiphenylsilyl-2-O-methansulfonyl)-propan-1-yl]-2-O-(2,3,4,6-tetra-O-benzyl-α-D-mannopyranosyl)-L-myo-inositol

In the same manner as described for 40-1 compound 56 (1.74 g, 1.24 mmol) gave 57 (1.77 g) in quantitative yield as a colourless syrup. −[α]_{D}=+18 (c=1, CHCl₃). —¹H-NMR (600 MHz, CDCl₃) 8 0.99 (s, 9H, tBu), 2.78 (s, 3H, OMs), 3.08 (dd, 1H, J_{1,2}=2.22 Hz, J_{1,6}=9.82 Hz, 1-H_{Inositol}), 3.21-3.29 (m, 2H, 5-H_{Inositol}, 3-H_{Inositol}), 3.46 (dd, 1H, J_{gem.}=9.71, 6-H_{Man}), 3.51 (dd, 1H, J_{6,5}=J_{6,1}=12.5 Hz, 6-H_{Inositol}), 3.61 (m, 1H, 6-H_{Man}), 3.66 (dd, 1H, J_{4,5}=J_{4,3}=10.08 Hz, 4-H_{Inositol}), 3.69 (m, 1H, ²-H_{Man}), 3.73 (s, 3H, OMe), 3.75-3.84 (m, 3H, 3″, 3-H_{Man}), 3.89-3.95 (m, 1H, 1″), 4.0-4.13 (m, 3H, 1″, 4H_{Man,} 5-H_{Man}), 4.29-4.89, (m, 18H, 2″, CH₂-Ph, 2-Hinosit₀i), 5.42 (d, 1H, 1-H_{Man}), 6. 69-6.77 (m, 2H, H_{PMB}), 7.03-7.41, 7.53-7.66 (m, 47H, ArH). —¹³C-NMR (150.9 MHz, CDCl₃) δ 19.13 (1C, C(CH₃)₃), 27.74 (3C, C(CH₃) ₃), 38.42 (1C, SO₂—CH₃), 55.20 (1C, OMe), 63.68 (1C, 3′-C), 69.08 (1C, 6-C_{Man}), 71.12 (1C, 2-C_{Inositol}), 71.72-77.3 (13C, CH₂Ph, 1′-C, 1-C_{Inositol}, 4-C_{Man,} 5-C_{Man,} 2-C_{Man}), 79.15 (1C, 3-C_{Man}), 80.87 (1C, 3-C_{Inositol}), 81.26 (1C, 4-C_{Inositol}), 81.79 (1C, 6-C_{Inositol}), 82.44 (1C, 2′-C), 83.09 (1C, 5-C_{Inositol}) 98.24 (1C, 1-C_{Man}), 113.76 (2C, C_{PMB}), 127.41-138.74 (57C, Ph), 159.12 (1C, C_{OMe}). - Anal. calcd. for C₈₉H₉₈O₁₄SSi (1483.9): C 72.00, H 6.66; found C 71.38, H 6.69.

### St 60: (+)-3,4,5-Tri-O-benzyl-1-O-(4-methoxybenzyl)-6-O-[(2S)-2-azido-3-O-(tert-butyldiphenylsilyl)-propan-1-yl]-2-O-(2,3,4,6-tetra-O-benzyl-α-D-mannopyranosyl)-L-myo-inositol

In the same manner as described for 41-1 compound 57 (1.77 g, 1.19 mmol) gave 60 (1.5 g, 88%) as a colourless syrup. −[α]_{D}=+12.7 (c=1, CHCl₃). —¹H-NMR (600 MHz, CDCl₃) δ 1.02 (s, 9H, tBu), 3.13 (dd, 1H, J_{1,2}=2.32 Hz, J_{1,6}=9.77 Hz, 1-H_{Inositol}), 3.25-3.30 (m, 2H, 5-H_{Inositol}, 3-H_{Inositol}), 3.36 (dd, 1H, J_{gem.}=10.6 Hz, 6-H_{Man}), 3.46 (dd, 1H, J_{6,5}=J_{6,1}=9.46 Hz, 6-H_{Inositol}), 3.5-3.79 (m, 11H, 6-H_{Man,} OMe, 1″, 2″, 3″, 4-H_{Inositol,} 2-H_{Man,} 3-H_{Man}), 3.84 (m, 1H, 1″), 4.01-4.06 (m, 2H, 4-H_{Man,} 5-H_{Man}), 4.29-4.66, 4.69-4.86 (m, 17H, CH₂-Ph, 2-H_{Inositol}), 5.39 (d, 1H, 1-H_{Man}), 6.68-6.76 (m, 2H, H_{PMB}), 7.06-7.41, 7.56-7.65 (m, 47H, ArH). —¹³C-NMR (150.9 MHz, CDCl₃) δ 19.09 (1C, C(CH₃)₃), 26.69 (3C, C(CH₃)₃), 55.19 (1C, OMe), 63.65 (1C, 3′-C), 64.57 (1C, 2′-C), 68.86 (1C, 6-C_{Man}), 71.5-76.02 (13C, CH₂Ph, 1′-C, 2-C_{Inositol,} 4-C_{Man,} 5-C_{Man,} 2-C_{Man}), 77.91 (1C, 1-C_{Inositol}), 79.19 (1C, 3-C_{Man}), 80-91 (1C, 3-C_{Inositol}), 81.24 (1C, 4C_{Inositol}) 82.01 (1C, 6-C_{Inositol}), 83.18 (1C, 5-C_{Inositol}), 98.35 (1C, 1-C_{Man}), 113.69 (2C, C_{PMB}), 127.37-138.81 (57C, Ph), 159.0 (1C, C_{OMe}). - Anal. calcd. for C₈₈H₉₅O₁₃N₃Si (1430.82): C 73.87, H 6.69, N 2.9; found C 73.63, H 6.78, N 2.35.

### St 61: (+)-3,4,5-Tri-O-benzyl-1-O-(4-methoxybenzyl)-6-O-[(2R)-2-azido-3-hydroxy-propan-1-yl]-2-O-(2,3,4,6-tetra-O-benzyl-α-D-mannopyranosyl)-L-myo-inositol

In the same manner as described for 43-1 compound 60 (1.47 g, 1.03 mmol) gave 61 (1.23 g, 92%) as a colourless syrup. −[α]_{D}=+15.6 (c=1, CHCl₃). —¹H-NMR (600 MHz, CDCl₃) δ 2.15 (s, 1H, OH), 3.17 (dd, 1H, J_{1,2}=1.91 Hz, J_{1,6}=9.87 Hz, 1-H_{Inositol}), 3.26-3.36 (m, 2H, 3-H_{Inositol}, 5-H_{Inositol}), 3.38-3.69 (m, 7H, 3″, 2″, 6H_{Man}, 4-H_{Inositol}, 6-H_{Inositol}), 3.71 (m, 1H, ²-H_{Man}), 3.75-3.83 (m, 4H, OMe, 3-H_{Man}), 3.85-3.95 (m, 2H, 1″), 4.01-4.09 (m, 2H, 4H_{Man,} 5-H_{Man}), 4.32-4.71, 4.72-4.88 (m, 17H, CH₂-Ph, 2-H_{Inositol}), 5.42 (d, 1H, 1-H_{Man}), 6.75-6.84 (m, 2H, H_{PMB}), 7.04-7.35 (m, 37H, ArH). —¹³C-NMR (150.9 MHz, CDCl₃) δ 55.25 (1C, OMe), 62.06 (1C, 3′-C), 62.65 (1C, 2′-C), 68.95 (1C, 6-C_{Man}), 71.09-76.0 (13C, CH₂Ph, 1′-C, 2-C_{Inositol,} 4C_{Man,} 5-C_{Man,} 2-C_{Man}), 77.46 (1C, 1-C_{Inositol}), 79.1 (1C, 3C_{Man}), 80.80 (1C, 3-C_{Inositol}), 81.34 (1C, 4-C_{Inositol}), 81-73 (1C, 6-C_{Inositol}), 82.29 (1C, 5-C_{Inositol}), 98.45 (1C, 1C_{Man}), 113.83 (2C, C_{PMB}), 127.4-138.61 (45C, Ph), 159.21 (1C, C_{OMe}). - Anal. calcd. for C₇₂H₇₇O₁₃N₃*½ H₂O (1201.43): C 71.98, H 6.54, N 3.5; found C 71.96, H 6.38, N 2.97.

### St 29: (+)-6-O-Allyl-3,4,5,-tri-O-benzyl-1-O-(4-methoxybenzyl)-2-O-(2,3,4,6-tetra-O-benzyl-α-D-mannopyranosyl)-(1→2) -L-myo-inositol

In the same manner as described for 36 compound 24 (15.9 g, 15.24 mmol) gave 29 (17 g, 98%) as a colourless syrup. −[α]_{D}=+20.1 (c=1, CHCl₃). —¹H-NMR (250 MHz, CDCl₃) δ 3.21 (dd, 1H, J=9.8 Hz, J=2.5 Hz, H_{Inositol}), 3.29-3.45 (m, 3H), 3.51-3.87 (m, 5H), 3.77 (s, 3H, OMe), 4.0-4.19 (m, 2H), 4.20-4.91 (m, 19H, CH₂-Ph, CH₂CH═CH₂), 2-H_{Inositol}), 5.12 (dd, 1H, J_{gem.}=10.36 Hz, J_{vic.}=1.68 Hz, CH₂CH═CH₂), 5.25 (dd, 1H, J_{gem.}=17.21 Hz, J_{vic.}=1.72 Hz, CH₂CH═CH₂), 5.42 (d, 1H, J=1.21 Hz, 1-H_{Man}), 5.88-6.04 (m, 1H, CH₂CH═CH₂), 6.75-6.84 (m, 2H, H_{PMB}), 7.07-7.40 (m, 37H, ArH). - Anal. calcd. for C₇₂H₇₆O₁₂ (1133.39): C 76.3, H 6.76; found C 76.1, H 6.57.

### St 31: (+)-3,4,5-tri-O-benzyl-1-o-(4-methoxybenzyl)-2-O-(2,3,4, 6-tetra-O-benzyl-α-D-mannopyranosyl)-(1->2) -L-myo-inositol

Compound 29 (7.74 g, 6.83 mmol) was dissolved in EtOH 90° C. (150 ml) by heating, then DBU (0.12 ml, 0.80 mmol) and (Ph₃P)₃RhCl (0.91 g, 1.02 mmol) were added. The mixture was stirred for 1.5 h under reflux and then concentrated in vacuo (propenyl, R_{f}=0.73, petrol ether/EtOAc 2:1). The residue was dissolved in 1:9 1M HCl/acetone, and the solution was heated under reflux for 20 min. Acidity was neutralized by adding triethylamine, then the mixture was diluted with ethyl acetate, washed with water, dried (MgSO₄) and concentrated. Flash chromatography (petrol ether/EtOAc 4:1) of the residue gave 31 (6.7 g, 90%). R_{f} (petrol ether/EtOAc 2:1)=0.61. −[α]_{D}=+28.3 (c=1, CHCl₃). —¹H-NMR (250 MHz, CDCl₃) δ 2.40 (d, 1H, J=1.75 Hz, OH), 3.01 (dd, 1H, J=2.4 Hz, J=9.98 Hz, H_{Inositol}), 3.32 (dd, 1H, J=9.17 Hz, H_{Inositol}), 3.34-3.40 (m, 1H, H_{Inositol}), 3.45-3.52 (m, 1H, 6-H_{Man}), 3.57-3.76 (m, 3H), 3.80 (s, 3H, OMe), 3.81-3.93 (m, 2H) 4.02 (dd, 1H, J=9.39 Hz), 4.10-4.19 (m, 1H, 5-H_{Man}), 4.38-4.91 (m, 17H, CH₂-Ph, 2-H_{Inositol}), 5.40 (d, 1H, J_{1,2}=1.50 Hz, 1-H_{Man}), 6.79-6. 86 (m, 2H, H_{PMB}), 7.10-7.40 (m, 37H, ArH). - Anal. calcd. for C₆₉H₇₂O₁₂ (1093.32): C 75.8, H 6.64; found C 75.51, H 6.56.

### St 63: (+)-3.4,5-Tri-O-benzyl-1-O-(4-methoxybenzyl)-6-O-[(2S)-2-azido-propionicacidbenzylester]-2-O-(2,3,4,6-tetra-O-benzyl-α-D-mannopyranosyl)-L-myo-inositol

In the same manner as described for 47-1 compound 61 (1.15 g, 0.96 mmol) gave 63 (1.15 g, 92%) as a colourless syrup. −[α]_{D}=+29.1 (c=1, CHCl₃). —¹H-NMR (250 MHz, CDCl₃) δ 3.15 (dd, 1H, J=9.69 Hz, H_{Inositol}), 3.25-3.40 (m, 3H), 3.55 (m, 2H), 3.63-3.85 (m, 3H), 3.76 (s, 3H, OMe), 3.93 (dd, 1H, J=3.9 Hz, J=6.11 Hz), 4.40-4.24 (m, 4H), 4.30-4.69, 4.70-4.90 (m, 17H, CH₂-Ph, 2-H_{Inositol}), 4.99 (d, 1H, J_{gem.}=12.22 Hz, COOCH₂-Ph), 5.20 (d, 1H, J_{gem.}=12.20 Hz, COOCH₂-Ph), 5.41 (d, 1H, J=1.39 Hz, 1-H_{Man}), 6.76-6.83 (m, 2H, H_{PMB}), 7.06-7.39 (m, 42H, ArH). - Anal. calcd. for C₇₉H₈₁O₁₄N₃ (1296.52): C 73.19, H 6.30, N 3.24; found C 72.87, H 6.35, N 2.95.

### St 64: (+)-3,4,5-Tri-O-benzyl-6-O-[(2S)-2-azido-propionicacidbenzylester]-2-O-(2,3,4,6-tetra-O-benzyl-α-D-mannopyranosyl)-L-myo-inositol

In the same manner as described for 48-1 compound 63 (1.1 g, 0.85 mmol) gave 64 (828 mg, 83%) as a colourless syrup. −[α]_{D}=+3.9 (c=1, CHCl₃). —¹H-NMR (250 MHz, CDCl₃) δ 2.83 (d, 1H, J=4.23 Hz, OH), 3.38-3.45 (m, 4H), 3.65-3.85 (m, 5H), 3.89-4.00 (m, 2H), 4.05-4.27 (m, 4H), 4.42-4.70, 4.71-4.92 (m, 14H, CH₂-Ph), 5.13 (d, 1H, J_{gem.}=12.14 Hz, COOCH₂-Ph), 5.22 (d, 1H, J_{gem.}=12.12 Hz, COOCH₂-Ph), 5.27 (d, 1H, J_{1,2}=1.48 Hz, 1-H_{Man}), 7.11-7.40 (m, 40H, ArH). - Anal. calcd. for C₇₁H₇₃O₁₃N₃ (1176.37): C 72.49, H 6.26, N 3.57; found C 72.32, H 6.27, N 3.15.

### St 65 a,b: (+)-3,4,5-Tri-O-benzyl-6-O-[(2S)-2-azido-propionicacidbenzylester]-2-O-(2,3,4,6-tetra-O-benzyl-α-D-mannopyranosyl)-L-myo-inosit-1-yl-[benzyloxy]-[(2R)-2,3-bis-(myristoyloxy)-propyl]-phosphate

In the same manner as described for 49-1 compound 64 (410 mg, 0.35 mmol) gave 65a,b (456 mg, 71%) as a colurless oil. −[α]_{D}=+7.6 (c=1, CHCl₃). —³¹P-NMR (600 MHz, CDCl₃) δ −0.1407 (s, 1P), 0.0651 (s, 1P). —¹H-NMR (600 MHz, CDCl₃) δ 0.78-0.93 (t, 12H, Me), 1.03-1.37 (s, 80H, CH₂-Chain), 1.44-1.65 (m, 8H, COCH₂CH₂R), 2.16-2.35 (m, 8H, COCH₂CH₂R), 3.30-0.40 (m, 4H, 3-H_{Inositol,} 5-H_{Inositol}), 3.53-3.87 (m, 12H, 6-H_{Inositol,} 4-H_{Inositol,} 3-H_{Man,} 2-H_{Man}), 3.90-4.00 (m, 6H, 6-H_{Inositol,} 5-H_{Inositol}) 4.03-4.28 (m, 14H, 6-H_{Man,} 1-H_{Inositol,} 4-H_{Inositol}), 4.34-4.90, (m, 30H, 2-H_{Inositol,} CH₂-Ph), 5.02-5.14, 5.16-5.28 (m, 10H, COOCH₂-Ph, POCH₂Ph), 5.30 (d, 1H, 1-H_{Man}), 5.37 (d, 1H, 1-H_{Man}), 7.02-7.44 (m, 90H, ArH). - MALDI: calcd. M+Sodium m/z=1864.4; found m/z=1863.8.

### St 66: 6-O-[(2R)-2-amino-propionicacid]-2-O-α-D-mannopyranosyl-L-myo-inosit-1-yl-[(2R)-2,3-bis-(myristoyloxy)-propyl]-phosphate

A vigorously stirred mixture of 65 (180 mg, 0.09 mmol), CH₂Cl₂:MeOH:H₂O (7.5:7.5:1, 3 ml) and Pearlman's catalyst (0.2 equiv.) was degassed under vacuum and saturated with hydrogen (by a H₂-filled balloon) three times. The suspension was stirred at room temperature over night, filtered over celite and washed with CH₂Cl₂:MeOH:H₂O (7.5:7.5:1, 2 ml). The solvents were removed under vacuum to afford 66 (83 mg, 85%) as a white solid. —³¹P-NMR (600 MHz, dmso) δ 0.023 (s, 1P). —¹H-NMR (600 MHz, dmso) δ 0.78-0.90 (t, 6H, Me), 1.08-1.35 (s, 40H, CH₂-Chain), 1.40-1.56 (m, 4H, COCH₂CH₂R), 2.18-2.33 (m, 4H, COCH+EE₂CH₂R), 3.12 (m, 1H, 5-H_{Inositol}), 3.23 (m, 1H, 3-H_{Inositol}) 3.24 (m, 1H, 6-H_{Man}), 3.27 (m, 1H, 4-H_{Man}), 3.30 (m, 1H, 6-H_{Inositol}), 3.38 (m, 1H, 4-H_{Inositol}), 3.47 (m, 1H, 3-H_{Man}), 3.65 (m, 1H, ²-H_{Man}), 3.66 (m, 1H, 1′), 3.69 (m, 1H, 6-H_{Man}), 3.78 (m, 1H, 2′), 3.8 (m, 1H, 1″/3″), 3.81 (m, 1H, 5-H_{Man}), 3.93 (m, 1H, 1″/3″), 3.94 (m, 1H, 2-H_{Inositol}), 3.99 (m, 1H, 1-H_{Inositol}), 4.06 (m, 1H, 1″/3″), 4.26 (m, 1H, 1′), 4.29 (m, 1H, 1″/3″), 5.02 (m, 1H, 1-H_{Man}), 5.10 (m, 1H, 2″), 8.65-8.9 (bs, 2H, NH₂). - Anal. calcd. for C₄₆H₈₆O₂₀NP*3/2 H₂O (1031.16): C 53.58, H 8.70, N 1.36; found C 53.61, H 8.71, N 0.80. - MALDI: calcd. (M−H⁺)^{−} m/z=1003.14; found m/z=1001.8, calcd. (M+Na)⁺ m/z=1027.16; found m/z=1027.5, calcd. [(M^{−}+Na⁺)Na]⁺ m/z 1050.16; found m/z=1049.4, calcd. [(M^{−}+Na⁺)K]⁺ m/z=1066.16; found m/z=1065.3.

### St 24: (+)-6-O-Allyl-3,4,5-tri-O-benzyl-1-O-(4-methoxybenzyl)-2-O-(2,4,6-tri-O-benzyl-α-D-mannopyranosyl)-(1→2) -L-myo-inositol

A mixture of imidate (20 g, 31.4 mmol), acceptor 20 (11.6 g, 19 mmol) were dissolved in anhydrous ether (220 ml). Trimethylsilyltriflate (0.52 ml) was added and the mixture was stirred for ten seconds, then quenched with triethylamine, diluted with toluene and concentrated. The residue was purified by flash chromatography (petrol ether/EtOAc 5:1→4:1) to gave crude product. TLC: (petrol ether/EtOAc 2:1, 1% NEt₃), R_{f}=0.72. Without further purification the crude product was dissolved in 200 ml of methylamine solution (33% in anhydrous EtOH) and stirred for 6 h at room temperature. The reaction mixture was concentrated, diluted with toluene and evaporated. Silica gel column chromatography of the residue (petrol ether/EtOAc 3:1→2:1) afforded 24 (14 g, 71% over 2 steps). R_{f} (petrol ether/EtOAc 3:1)=0.40. −[α]_{D}=+41 (c=1, CHCl₃). —¹H-NMR (600 MHz, CDCl₃) δ 2.38 (s, 1H, OH), 3.20 (dd, 1H, J_{1,2}=2.0 Hz, J_{1,6}=9.88 Hz, 1-H_{Inositol}), 3.28-3.40 (m, 3H, 5-H_{Inositol}, 3-H_{Inositol}, 6-H_{Man}), 3.50 (dd, 1H, J_{gem.}=10.67 Hz, J_{vic.}=3.2 Hz, 6-H_{Man}), 3.69 (dd, 1H, J_{6,1}=J_{6,5}=9.54 Hz, 6-H_{Inositol}), 3.76 (s, 3H, OMe), 3.78-3.86 (m, 2H, 4-H_{Inositol}, 3-H_{Man}), 3.92 (dd, 1H, J_{4,5}=J_{4,3}=9.65 Hz, 4-H_{Man}), 4.06 (s, 1H, 2-H_{Man}), 4.12-4.17 (m, 1H, 5-H_{Man}), 4.25-4.31 (m, 1H, CH₂=CH—CH₂), 4.32 (m, 1H, 2-H_{Inositol}), 4.34-4.40, 4.5-4.56, 4.57-4.73, 4.74-4.88 (m, 15H, CH₂-Ph, CH₂=CH—CH₂), 5.13 (dd, 1H, J_{gem.}=10.32 Hz, CH₂=CH—CH₂), 5.25 (dd, 1H, CH₂=CH—CH₂), 5.38 (d, 1H, J<1, 1-H_{Man}), 5.89-6.0 (m, 1H, CH₂=CH—CH₂), 6.72-6.81 (m, 2H, H_{PMB}), 7.06-7.38 (m, 32H, ArH). —¹³C-NMR (150.9 MHz, CDCl₃) δ 55.20 (1C, OMe), 68.55 (1C, ⁶-C_{Man}), 68.65 (1C, 2-C_{Man}), 70.90 (1C, 5-C_{Man}), 72.10-76.19 (9C, CH₂-CH═CH₂, CH₂Ph, 4-C_{Man}), 78.44 (1C, 1-C_{Inositol}), 79.61 (1C, ³-C_{Man}), 80.75 (1C, 3-C_{Inositol}), 81.04 (1C, 6-C_{Inositol}), 81.42 (1C, 4-C_{Inositol}), 83.44 (1C, 5-C_{Inositol}), 99.97 (1C, 1-C_{Man}), 113.65 (2C, C_{PMB}), 116.57 (1C, CH₂—═CH₂), 127.46-138.70 (40C, Ph, CH₂—═CH₂), 159.00 (1C, C_{OMe}). - Anal. calcd. for C₆₅H₇₀O₁₂ (1043.26): C 74.8, H 6.76; found C 74.92, H 6.51.

### St 26: (+)-3,4,5,6-Tetra-O-benzyl-1-O-(4-methoxybenzyl)-2-O- (2,3,4,6-tetra-O-benzyl-α-D-mannopyranosyl)-(1→2) -L-myo-inositol

To a solution of 25 (2.5 g, 2.49 mmol), in dry DMF (50 ml), was added benzyl bromide (0.75 ml, 6.31 mmol) and sodium hydride (150 mg, 6.25 mmol). The reaction mixture was stirred at room temperature for 3 h, quenched with MeOH and concentrated. The residue was partitioned between ethyl acetate and water. The organic layer was washed with brine, dried (MgSO₄) and evaporated in vacuo. Silica gel column chromatography (petrol ether/ethyl acetate 4:1) afforded 26 (2.5 g, 86%) as a colourless syrup. R_{f} (petrol ether/ethyl acetate 4:1)=0.27. −[α]_{D}=+20 (c=1, CHCl₃). —¹H-NMR (250 MHz, CDCl₃) δ 3.23-3.49 (m, 4H), 3.59 (dd, 1H, J_{gem.}=10.6 Hz, J_{vic.}=3.5 Hz, 6-H_{Man}), 3.66-3.88 (m, 4H), 3.76 (s, 3H, OMe), 4.0-4.2 (m, 2H), 4.32-4.96 (m, 19H, CH₂-Ph, 2-H_{Inositol}), 5.42 (d, 1H, J=1.40 Hz, 1-H_{Man}), 6.70-6.80 (m, 2H, H_{PMB}), 7.09-7.42 (m, 42H, ArH). - Anal. calcd. for C₇₆H₇₈O₁₂ (1183.5): C 77.1, H 6.64; found C 77.06, H 6.73.

### St 27: (+)-3,4,5,6-Tetra-O-benzyl-2-O-(2,3,4,6-tetra-O-benzyl-α-D-mannopyranosyl)-(1→42)-L-myo-inositol

A solution of 26 (2.6 g, 2.20 mmol), in acetonitrile:toluene:water (60:3:4, 3 ml), was cooled to 0° C. and treated with Ce(NH₄)₂(NO₃)₆(6 g, 10.94 mmol). After ½ at 0° C., the reaction was allowed to reach room temperature. The mixture was stirred for 1.5 h, diluted with EtOAc, washed with saturated aqueous NaHCO₃-solution, dried (MgSO₄) and concentrated. Flash chromatography (petrol ether/EtOAc 4:1→3:1) of the residue gave 27 (2.2 g, 94%) as a colourless syrup. TLC: (petrol ether/EtOAc 5:2), R_{f}=0.26. −[α]_{D}=+10.8 (c=1, CHCl₃). —¹H-NMR (250 MHz, CDCl₃) δ 2.45 (s, 1H, OH), 3.37-3.49 (m, 3H), 3.53-3.81 (m, 6H), 3.90 (dd, 1H, J=8.7 Hz), 4.01-4.11 (m, 1H), 4.21 (dd, 1H, J_{2,1}=J_{2,3}=2.3 Hz, 2-H_{Inositol}), 4.42-4.93 (m, 16H, CH₂-Ph), 5.22 (d, 1H, J=1.20 Hz, 1-H_{Man}), 7.13-7.41 (m, 40H, ArH). - Anal. calcd. for C₆₈H₇₀O₁₁*H₂O (1081.3): C 75.53, H 6.71; found C 75.48, H 6.50. - MALDI: calcd. (M+Na)⁺ m/z=1086.3; found m/z=1086.8.

### St 28 : 3,4,5,6-Tetra-O-benzyl-2-O-(2,3,4,6-tetra-O-benzyl-α-D-mannopyranosyl)-L-myo-inosit-1-yl-[(2R)-2,3-bis-[(myristoyloxy)-propyl]-phosphate

Terazole (172 mg, 2.46 mmol) was dried for 1 h under high vacuum. Phosphitamide (1 g, 0.94 mmol) was dissolved in anhydrous dichloromethane (50 ml), added to the tetrazole and stirred at rt under argon atmosphere. To this reaction mixture was added dropwise benzyl N,N-diisopropylphosphoramidite (1.35 g, 1.89 mmol) and stirred for 2 h. Then treated with tertbutylperoxide in isooctane (4.7 M, 3.76 ml). After 15 min, the reaction mixture was concentrated to 10 ml, treated with dimethylamine solution (20 ml, 33% in anhydrous EtOH) and stirred for 1 h. Then the reaction mixture was again concentrated to 10 ml, diluted with CH₂Cl₂, saturated NaHCO₃-solution was added and the two layers were seperated. The organic layer was washed with brine, dried (MgSO₄) and concentrated. The residue was purified by flash chromatography (toluene/acetone 9:1→1:10) to afford 29 (1.17 g, 76%) as a colourless syrup. —³¹P-NMR [600 MHz, CD₃OD/CDCl₃ (1:1)] δ −2.927 (s, 1P). —¹H-NMR [600 MHz, CD₃OD/CDCl₃ (1:1)] δ 0.89 (t, 6H, Me), 1.1-1.40 (s, 40H, CH₂-Chain), 1.45-1.60 (m, 4H, COCH₂CH₂R), 2.15-2.30 (m, 4H, COCH₂CH₂R), 3.40-3.54 (m, 2H, 3-H_{Inositol,} 5-H_{Inositol}), 3.67 (dd, 1H, J_{4,5}=J_{4,3}=9.44 Hz, 4-H_{Inositol}), 3.50-3.58 (m, 2H, 2′, 6-H_{Inositol}), 3.59-3.84 (m, 1H, 3′), 3.87-3.99 (m, 4H, 1″, 3″, 6-H_{Man}), 4.0-4.07, (m, 2H, 1-H_{Inositol,} 1″), 4.09-4.18 (m, 2H, 4-H_{Man,} 5-H_{Man}), 4.19-4.25 (m, 1H, 3″), 4.38-4.93 (m, 17H, CH₂-Ph, 2-H_{Inositol}), 5.14-5.20 (m, 1H, 2″), 5.31 (d, 1H, J<1 Hz, 1-H_{Man}), 7.10-7.42 (m, 40H, ArH). - MALDI: calcd. (M−H)⁻ m/z=1636.15; found m/z=1635.8. - FAB-MS: (M⁻Na⁺)Na⁺ m/z 1684; found m/z=1684

### St 30: Triethylammonium-[2-O-(α-D-mannopyranosyl)-L-myo-inosit-1-yl]-[(2R)-2,3-bis(myristoyloxy)propyl]-phosphate

A vigorously stirred mixture of 28 (394 mg, 0.24 mmol), CH₂Cl₂:MeOH:H₂O (7.5:7.5:1, 5 ml) and Pearlman's catalyst (0.2 equiv.) was degassed under vacuum and saturated with hydrogen (by a H₂-filled balloon) three times. The suspension was stirred at room temperature over night, filtered over celite and washed with CH₂Cl₂:MeOH:H₂O (7.5:7.5:1, 2 ml). The solvents were removed under vacuum to afford 30 (201 mg, 91%) as a white solid. ³¹P-NMR (600 MHz, dmso) 8 0.963 (s, 1P). —¹H-NMR (600 MHz, dmso) δ 0.76-0.91 (t, 6H, Me), 1.0-1.40 (m, 49H, CH₂-Chain, Me_{NEt3}), 1.41-1.57 (m, 4H, COCH₂CH₂R), 2.15-2.32 (m, 4H, COCH₂CH₂R), 2.85-2.95 (m, 1H, 5-H_{Inositol}), 2.96-3.15 (m, 6H, HN(CH₂—CH₃)₃), 3.22 (m, 1H, 4-H_{Man}), 3.23 (m, 1H, 6-H_{Man}), 3.24 (m, 1H, 3-H_{Inositol}), 3.34, (m, 1H, 4-H_{Inositol}), 3.45 (m, 1H, 3-H_{Man}), 3.46 (m, 1H, 6-H_{Inositol}), 3.61 (m, 1H, 1-H_{Inositol}), 3.64 (m, 1H, ²H_{Man}), 3.72 (m, 1H, ⁶H_{Man}), 3.76 (m, 1H, 1″), 3.9 (m, 1H, 1″), 4.00 (m, 1H, 2-H_{Inositol}), 4.06 (m, 1H, 3″), 4.08 (m, 1H, 5-H_{Man}), 4.28 (m, 1H, 3″), 4.95 (m, 1H, 1-H_{Man}), 5.09 (m, 1H, 2″). - MALDI: calcd. (M−H⁺)⁻ m/z=915.67; found m/z=915.9. - FAB-MS: (M−H⁺)⁻ m/z=915.67; found m/z=915.

References:

The references cited herein are all expressly incorporated by reference.
1. Vacca et al, Tetrahedron, 1989, 45, 5679-5702.
2. Aguiló et al, Tetrahedron Letters, 1992, 33, 401-404.
3. Anderson, The Carbohydrates 1A, W. Pigman, D. Horton: New York, 1972, p 519.
4. Plouvier, Bull. Soc. Chim. Biol., 1963, 45, 1079.
5. Angyal & Bender, J. Chem. Soc., 1961, 4718.
6. Post & Anderson, J. Amer. Chem. Soc., 1962, 84, 471, 478.
7. Moon Kim & Sharpless, Tetrahedron Letters, 1989, 30, 655-658.
8. Gao & Sharpless, J. Am. Chem. Soc., 1988, 110, 7538-7539.
9. Klotz, Dissertation, 1994, Universität Konstanz.
10. Sato et al, J. Org. Chem., 1992, 57, 2166-2169.
11. Mayer, Dissertation 1996, Universität Konstanz
12. Schmidt & Michel, Angew. Chem., 1980, 92, 763-764; Angew. Chem. Int. Ed. Engl., 1980, 19, 731-732.
13. Bannwarth & Trzeciak, HeIv. Chim. Acta 1987, 70,175-186.
14. Beaucage & Iyer, Tetrahedron 1993, 49,10441-10488.
15. Beaucage & Caruthers, Tetrahedron Lett. 1981, 22, 1859-1862.
16. Maki et al, Tetrahedron Lett., 1998, 39, 5601-5604
17. Van Rheenan et al, Org. Syn. Coll., 1988, VI, 342.

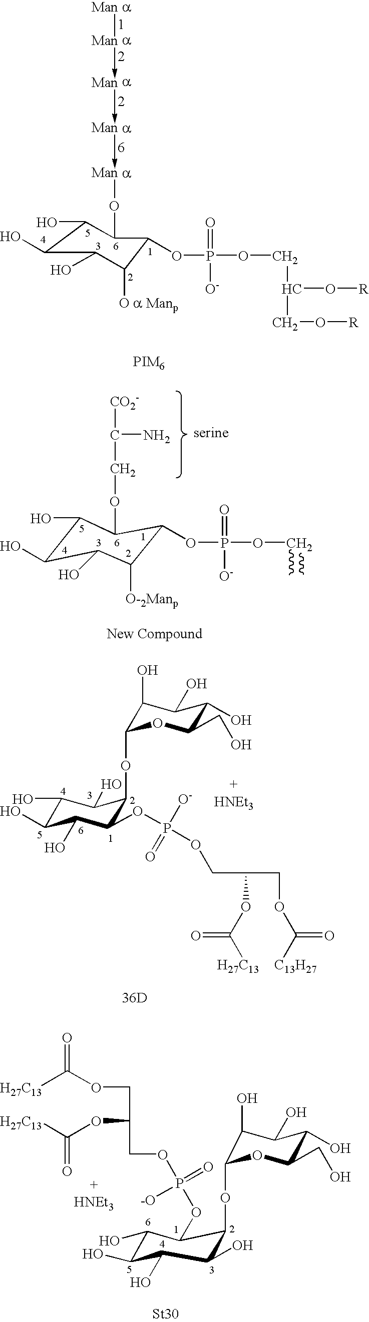

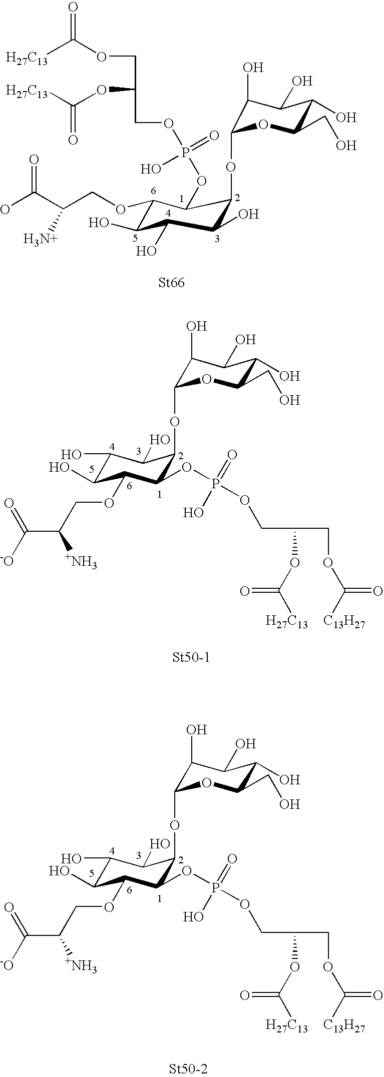

1. A compound comprising a myo-inositol which is substituted at position 1 with a phosphate ester group, at position 2 with a sugar group and at one or both of position 4 and y position 6 with an amino acid group, or a coupling partner or a derivative of the compound.

2. The compound of claim 1, wherein the compound is represented by one of the structural formulae: 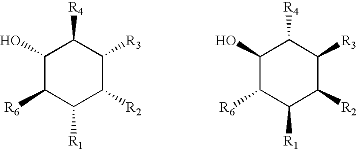
wherein:
R1 is hydroxyl, phosphate, phosphatidic acid or a phosphate ester;
R2 is a sugar moiety;
R3 is are selected from hydroxyl or phosphate; and, at least one of R4 R6 is independently selected from:
an amino acid; or
a peptide or polypeptide; or
a group having the general formula:
—O—(CH2)n—CH(NR7R8)-CO2X,
wherein:
n is an integer between 1 and 10,
R7 and R8 are independently selected from hydrogen, nitrogen, acyl or alkyl; and
X is hydrogen, alkyl or a cation where the terminal group is —CO2—; or
a substituted or unsubstituted aromatic group, such as a group represented by the general formula: 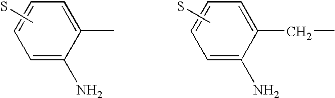
wherein S is hydrogen or one more aromatic substituents; and
wherein when one of R4 or R6 is as defined above, the other may be hydroxyl or phosphate; or a coupling partner or derivative thereof.

3. The compound of claim 2, wherein the R1 group is a phosphate ester group which is a phosphate lipid ester in which a phosphate group is linked to position 1 of the inositol ring.

4. The compound of claim 3, wherein the phosphate group is represented by the following formula, wherein Y is an alkyl group linked to one or more lipid groups:

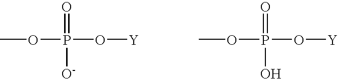

5. The compound of claim 3, wherein the phosphate ester comprises one or more lipidic groups selected from lyso, acyl, alkyl, diacylglyceryl, alkylacylglyceryl, dialkylglyceryl, ceramidyl, lysospingosine, acylglyceryl, or alkylglyceryl groups.

6. The compound of claim 2, wherein the sugar moiety (R2) at position 2 is a hexose.

7. The compound of claim 6, wherein the hexose is selected from glucosamine, galactosamine, galactose, mannose, glucose, fucose or xylose, or a substituted derivative thereof.

8. The compound of claim 1, wherein the derivative of the compound is a salt, a coordination complex with a metal ion, an ester, a free acid or a free base, a hydrate, a prodrug or a lipid.

9. The compound of claim 2, wherein, the R4 substituent at position 4 and/or the R6 substituent at position 6 is an amino acid or amino acid mimetic group or group for linking to a coupling partner.

10. The compound of claim 1, wherein the coupling partner is a peptide, polypeptide or carbohydrate for delivery to caveolae.

11. The compound of claim 1, wherein the coupling partner is a vaccine, a growth factor, or a receptor.

12. The compound of claim 11, wherein the amino acid is serine coupled at one or both of position 4 and position 6 of the inositol ring.

13. The compound of claim 1, wherein a polypeptide is coupled via a Ser-Ser linkage at position 4 and/or position 6 of the inositol ring so that the coupling partner can be enzymatically cleaved after delivery to caveolae.

14. The compound of claim 1 which is:
Triethylammonium-[2-O-(-D-mannopyranosyl)-D-myo-inosit-1-yl]-[(2R)-2,3-bis-(myristoyloxy)-propyl]-phosphate;
Triethylammonium-[2-O-(-D-mannopyranosyl)-L-myo-inosit-1-yl]-[(2R)-2,3-bis(myristoyloxy)propyl]-phosphate;
6-O-[(2R)-2-amino-propionic acid]-2-O-D-mannopyranosyl-L-myo-inosit-1-yl-[(2R)-2,3-bis-(myristoyloxy)-propyl]-phosphate;
6-O-[(2S)-2-amino-propionic acid]-2-O-D-mannopyranosyl-D-myo-inosit-1-yl-[(2R)-2,3-bis-(myristoyloxy)-propyl]-phosphate;
6-O-[(2R)-2-amino-propionic acid]-2-O-D-mannopyranosyl-D-myo-inosit-1-yl-[(2R)-2,3-bis-(myristoyloxy)-propyl]-phosphate.

15. The compound of claim 4, wherein the phosphate ester comprises one or more lipidic groups selected from lyso, acyl, alkyl, diacylglyceryl, alkylacylglyceryl, dialkylglyceryl, ceramidyl, lysospingosine, acylglyceryl, or alkylglyceryl groups.

16. The compound of claim 2, wherein the derivative of the compound is a salt, a coordination complex with a metal ion, an ester, a free acid or a free base, a hydrate, a prodrug or a lipid.

17. The compound of claim 2, wherein the coupling partner is a peptide, polypeptide or carbohydrate for delivery to caveolae.

18. The compound of claim 2, wherein the coupling partner is a vaccine, a growth factor, or a receptor.

19. The compound of claim 18, wherein the amino acid is serine coupled at position 4 and/or position 6 of the inositol ring.

20. The compound of claim 2, wherein a polypeptide is coupled via a Ser-Ser linkage at position 4 and/or position 6 of the inositol ring so that the coupling partner can be enzymatically cleaved after delivery to caveolae.

21. The compound of claim 2 which is:
Triethylammonium-[2-O-(-D-mannopyranosyl)-D-myo-inosit-1-yl]-[(2R)-2,3-bis-(myristoyloxy)-propyl]-phosphate;
Triethylammonium-[2-O-(-D-mannopyranosyl)-L-myo-inosit-1-yl]-[(2R)-2,3-bis(myristoyloxy)propyl]-phosphate;
6-O-[(2R)-2-amino-propionic acid]-2-O-D-mannopyranosyl-L-myo-inosit-1-yl-[(2R)-2,3-bis-(myristoyloxy)-propyl]-phosphate;
6-O-[(2S)-2-amino-propionic acid]-2-O-D-mannopyranosyl-D-myo-inosit-1-yl-[(2R)-2,3-bis-(myristoyloxy)-propyl]-phosphate;
6-O-[(2R)-2-amino-propionic acid]-2-O-D-mannopyranosyl-D-myo-inosit-1-yl-[(2R)-2,3-bis-(myristoyloxy)-propyl]-phosphate.
